(19) United States
(12) Patent Application Publication
(10) Pub. No.: US 2026/0079086 A1
(43) Pub. Date: Mar. 19, 2026

(54) APPARATUS, SYSTEM, AND METHOD FOR HIGH YIELD MAGNETIC SEPARATION OF A BIOLOGICAL POPULATION

(71) Applicant: OCTANE BIOTECH, INC., Kingston (CA)

(72) Inventors: Nuala Trainor, Kingston (CA); Michele Vicentini-Hogan, Kingston (CA); Perryn Bennett, Kingston (CA); Ashkan Tabibian, Kingston (CA); James Marshall, Kingston (CA); Carlene Petes, Kingston (CA); Chad Ibbotson, Kingston (CA)

(21) Appl. No.: 19/298,287

(22) Filed: Aug. 13, 2025

Related U.S. Application Data
(60) Provisional application No. 63/695,904, filed on Sep. 18, 2024.

(51) Int. Cl. G01N 1/34; C12N 15/10

(52) U.S. Cl. CPC G01N 1/34; C12N 15/1013

(57) ABSTRACT

The present disclosure provides systems, methods, and apparatuses for improved magnetic separation of a biological population. A system for magnetic separation and collection of a target biological population from a biological sample comprises a cell engineering cassette, and a fluidic pathway disposed within the cell engineering cassette. The fluidic pathway has entrapment features disposed along a flow path of the fluidic pathway. An array of magnets is disposed adjacent to the fluidic pathway and the cell engineering cassette such that the array of magnets can be translatable toward and away from the fluidic pathway to apply a magnetic field to the biological population for the separation of a first subpopulation from a second subpopulation.

### FIELD OF THE INVENTION

The present disclosure relates to devices, methods, and systems for automated magnetic separation of a target biological population from a biological sample. Such devices, methods and systems find use in a variety of clinical and laboratory settings.

### BACKGROUND

Magnetic separation has been utilized as a method to separate magnetic impurities from fluids through the application of a variety of different processes. Magnetic separation techniques have also been applied to the separation of populations of biological materials using magnetic beads that have been coated with antibodies or polymers to bind to various biological targets, including viruses, bacteria, and cells. The biological target can then be extracted from the fluid suspension using magnetic separation devices. The magnetic field generated in the separation device applies a force on the magnetic beads suspended within, which can draw the bead out of fluid suspension, as well as any biological material bound to the magnetic bead. This allows for the desired population to be isolated, by either removing it from the fluid suspension (known as positive selection), or by removing all other populations from the fluid suspension to leave only the non-magnetically bound population of interest (known as negative selection). Isolation of cells, such as T-cells, genetically modified T-cells, and stem cells, or more particularly targeted subpopulations of cells, from heterogeneous cell populations is necessary for the development of cell therapies used to treat a variety of diseases.

However, prior magnetic separation devices are inconsistent with their separation capabilities, as they can result in low cell yield, purity, and/or viability parameters. There thus remains an unmet need for rapid, consistent, and reliable magnetic separation of a selected target within a biological sample where the application of a magnetic field may be automated, customized, and controlled for separation of the target with a desired high yield and high purity. There further exists an unmet need for isolation of smaller magnetic particles (i.e., nano sized) due to concerns that larger magnetic particles (i.e., micron sized) may have an impact on the patient if they are implanted along with a cell therapy product. Presently, it is more challenging to separate smaller magnetic particles as they contain less magnetic materials, and thus require a more powerful magnetic separation system for effective isolation. Furthermore, the use of a more powerful magnetic separation system creates a problem to be solved concerning the effective release of larger magnetic particles captured via the magnetic separation system.

### SUMMARY OF THE INVENTION

In some aspects, the techniques described herein relate to a system for magnetic separation and collection of a target biological population from a biological sample, including: a cell engineering cassette; a fluidic pathway disposed within the cell engineering cassette, the fluidic pathway having entrapment features disposed along a flow path of the fluidic pathway; and an array of magnets disposed adjacent to the fluidic pathway and to the cell engineering cassette such that the array of magnets can be translatable toward and away from the fluidic pathway.

In some aspects, the techniques described herein relate to a method for collecting a biological population from a biological sample having at least a first subpopulation and a second subpopulation, including: binding the first subpopulation to a plurality of magnetic particles; flowing the biological sample through a flow path of a fluidic pathway having entrapment features disposed therein; positioning an array of magnets such that the fluidic pathway is exposed to a magnetic field generated by the array of magnets; exposing the biological population to the magnetic field; entrapping the first subpopulation bound to the plurality of magnetic particles to the entrapment features and/or a sidewall of the fluidic pathway; removing and collecting the second subpopulation from the fluidic pathway; positioning the array of magnets such that the fluidic pathway is not exposed to the magnetic field; removing the first subpopulation bound to the plurality of magnetic particles from the fluidic pathway; and collecting the first subpopulation bound to the plurality of magnetic particles.

In some aspects, the techniques described herein relate to a fluidic pathway for flowing a biological sample and magnetic particles along a flow path therein, including: a fluidic pathway having a height of approximately 5-44 mm, a width of approximately 0.5-6 mm, and a length of approximately 50-520 mm; and entrapment features disposed along the length of the fluidic pathway, each of the entrapment features have a height and/or a width of about 0.05-1 mm; wherein the entrapment features are configured to decrease a flow velocity of a first subpopulation of the biological sample, wherein the first subpopulation is bound to a plurality of magnetic particles.

In some aspects, the techniques described herein relate to a system for magnetic separation and collection of a target biological population from a biological sample, including: a cell engineering cassette; a fluidic pathway disposed within the cell engineering cassette; and an array of magnets disposed adjacent to the fluidic pathway and to the cell engineering cassette, the array of magnets configured for engaging the fluidic pathway with a magnetic field when in an ON position, and configured for disengaging, disrupting, or blocking the magnetic field from the fluidic pathway when in an OFF position.

### BRIEF DESCRIPTION OF THE DRAWINGS

The following description of typical aspects described herein will be better understood when read in conjunction with the appended drawings. For the purpose of illustrating the invention, there are shown in the drawings aspects which are presently typical. It should be understood, however, that the invention is not limited to the precise arrangements and instrumentalities of the aspects shown in the drawings. It is noted that like reference numerals refer to like elements across different embodiments as shown in the drawings and referred to in the description.

FIG. 1A illustrates an embodiment of a magnetic separation system applied to a flow through tubing concept as further described herein.

FIG. 1B illustrates an embodiment of the magnetic separation system applied to a flow through tubing concept comprising a fluidic pathway as further described herein.

FIG. 2A illustrates an embodiment of a cell engineering cassette including an array of magnets disposed on a satellite chamber of a cell engineering cassette as further described herein.

FIG. 2B illustrates an embodiment of the magnetic separation system including an array of magnets disposed on a satellite chamber of a cell engineering cassette as further described herein.

FIG. 2C illustrates the magnetic separation system of FIG. 2B, with the array of magnets swiveled away from a biological sample as further described herein.

FIG. 3A illustrates an embodiment of the cell engineering cassette with the array of magnets disposed on a proliferation chamber as further described herein.

FIG. 3B illustrates a side elevational view of the cell engineering cassette of FIG. 3A.

FIG. 3C illustrates a perspective view of an embodiment of a cell engineering cassette with the array of magnets disposed on a proliferation chamber as further described herein.

FIG. 3D illustrates another perspective view of the cell engineering cassette with the array of magnets disposed on a proliferation chamber of FIG. 3C.

FIG. 4 illustrates an embodiment of the cell engineering cassette with the array of magnets disposed on a crossflow reservoir as further described herein.

FIG. 5A illustrates an embodiment of the cell engineering cassette with the array of magnets disposed on a container module as further described herein.

FIG. 5B illustrates an embodiment of the cell engineering cassette and the array of magnets disposed on the container module of FIG. 5A.

FIG. 6A illustrates a front elevational view of an embodiment of the magnetic separation system with the array of magnets disposed on a warm zone of the magnetic separation system as further described herein.

FIG. 6B illustrates an embodiment of the magnetic separation system with the array of magnets disposed on a warm zone of the magnetic separation system as further described herein.

FIG. 7A illustrates an embodiment of the magnetic separation system with the array of magnets disposed on a fluid reservoir and/or a cold zone as further described herein.

FIG. 7B illustrates an embodiment of the magnetic separation system with the array of magnets disposed in a cold zone as further described herein.

FIG. 7C illustrates an embodiment of the magnetic separation system with the array of magnets disposed on or within a cold zone as further described herein.

FIG. 7D illustrates an embodiment of the magnetic separation system with the array of magnets disposed within a cold zone as further described herein.

FIG. 8 is a chart illustrating the various locations the array of magnets and/or fluidic pathway may be placed on or within the magnetic separation system and/or the cell engineering cassette as further described herein.

FIG. 9A illustrates an embodiment of the array of magnets as further described herein.

FIG. 9B illustrates an embodiment of the array of magnets and the magnetic field emitted therefrom.

FIG. 9C is a front elevational view illustrating the array of magnets and the magnetic field emitted therefrom.

FIG. 10A illustrates embodiments of the array of magnets for application to the embodiments of the magnetic separation system as further described herein.

FIG. 10B illustrates embodiments of the array of magnets as further described herein.

FIG. 11 illustrates the array of magnets affixed to a swivel component as further described herein.

FIG. 12 illustrates embodiments of magnetic particles for use with the magnetic separation system as further described herein.

FIG. 13 illustrates embodiments of the magnetic particles for use with the magnetic separation system, and the optical magnetic field gradient implementations applied thereto, as further described herein.

FIG. 14A illustrates embodiments of the fluidic pathway of the magnetic separation system as further described herein.

FIG. 14B illustrates a partial side cross-sectional view of the fluidic pathway and entrapment features of the magnetic separation system of FIG. 14A.

FIG. 15 illustrates a flowchart of a method of magnetic separation as further described herein.

FIG. 16 illustrates test results of magnetic separation performance with flow through tubing.

FIG. 17 illustrates test results of magnetic separation performance with a backward compatibility application.

FIG. 18 illustrates test results of magnetic separation performance with an array of magnets and fluidic pathway as further described herein.

### DETAILED DESCRIPTION OF THE INVENTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques).

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element.

The terms “invention” or “present invention” are non-limiting terms and are not intended to refer to any single aspect of the particular invention, but encompass all possible aspects as described in the specification and the claims.

The use of the term “or” in the claims is used to mean “and/or,” unless explicitly indicated to refer only to alternatives or the alternatives are mutually exclusive, although the disclosure supports a definition that refers to only alternatives and “and/or.” As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” means that the specific terms recited are representative examples and embodiments of the disclosure that are not intended to be limited to the specific examples referenced or cited unless explicitly stated otherwise.

As used herein, “about” can mean plus or minus 10% of the provided value. Where ranges are provided, they are inclusive of the boundary values. “About” can additionally or alternately mean either within 10% of the stated value, or within 5% of the stated value, or in some cases within 2.5% of the stated value; or, “about” can mean rounded to the nearest significant digit.

As used herein, the terms “close”, “approximate”, and “practically” denote a respective relation or measure or amount or quantity or degree that has no adverse consequence or effect relative to the referenced term or embodiment or operation of the scope of the invention.

As used herein, “between” is a range inclusive of the ends of the range. For example, a number between x and y explicitly includes the numbers x and y and any numbers that fall within x and y.

As may be used herein any terms referring to geometrical relationships such as “vertical”, “horizontal”, “parallel”, “opposite”, “straight”, “lateral”, “parallel”, “perpendicular”, and other angular relationships denote also approximate yet functional and/or practical, respective relationships.

As may be used herein, the terms “preferred”, “preferably”, “typical”, “typically”, or “optionally” do not limit the scope of the invention or embodiments thereof.

As may be used herein, the term “biological sample” may be any material derived from a human or other specimen. As described herein, a biological sample may comprise a body fluid sample, a body cell sample, an in-vitro cell sample, a genetically engineered cell sample, or a biological tissue sample. Examples of biological samples include urine, lymph, blood, plasma, serum, saliva, cervical fluid, cervical-vaginal fluid, vaginal fluid, breast fluid, breast milk, synovial fluid, semen, seminal fluid, stool, sputum, cerebral spinal fluid, tears, mucus, interstitial fluid, follicular fluid, amniotic fluid, aqueous humor, vitreous humor, peritoneal fluid, ascites, sweat, lymphatic fluid, lung sputum and lavage or samples derived therefrom. Biological tissue samples are samples containing an aggregate of cells, usually of a particular kind, together with intracellular substances that form one of the structural materials of a human, animal, plant, bacterial, fungal or viral structure, including connective, epithelium, muscle and nerve tissues. Examples of biological tissue samples also include organs, tumors, lymph nodes, arteries and individual cell(s). for example, the sample can be a tissue sample suspected of being cancerous. Biological tissue samples may be first treated to separate aggregates of cells.

In embodiments, the biological sample is a blood cell, white blood cell or platelet. White blood cells (leukocytes) include neutrophils, lymphocytes (T cells inclusive of T helper cells, cytotoxic T cells, T-killer cells, Natural Killer, and B lymphocytes), monocytes, eosinophils, basophils, macrophages, and dendritic cells. The biological sample may include peripheral blood mononuclear cells (PBMC), such as T cells, monocytes, natural killer cells, and/or dendritic cells.

As used herein, “biological population” is a subset of a biological sample, or a subsample thereof, as derived from a human or other specimen. A biological population may include a collection, subset, or subpopulation of cells or other biological materials derived from urine, lymph, blood, plasma, serum, saliva, cervical fluid, cervical-vaginal fluid, vaginal fluid, breast fluid, breast milk, synovial fluid, semen, seminal fluid, stool, sputum, cerebral spinal fluid, tears, mucus, interstitial fluid, follicular fluid, amniotic fluid, aqueous humor, vitreous humor, peritoneal fluid, ascites, sweat, lymphatic fluid, lung sputum and/or lavage. A biological population which may be a “target biological population” can include cells, nucleic acids, proteins, peptides or other biologic structures. The biological population may include a collection or subsample of peripheral blood mononuclear cells (PBMC), such as T cells, monocytes, natural killer cells, and/or dendritic cells.

As used herein, “target cells” are cells typically intended for separation or concentration from other cells (such as for examination or diagnosis), of particular type or having distinct characteristics relative to other cells, such as selective mutual affinity to couple with certain antibodies or other compounds or other particles. In particular embodiments, a distinct characteristic is selective affinity to couple or bind with magnetic beads to form magnetic target cells. The cells not identified as “target cells” may be identified as “non-target cells” as used herein.

As used herein, the term “patient sample” is defined as a biological sample taken from any animal for whom diagnosis, screening, monitoring or treatment is contemplated. Animals include mammals. A patient refers to a subject such as a mammal, primate, human or livestock subject afflicted with a disease condition or for which a disease condition is to be determined or treated. A patient sample may be the source of a source biological sample.

As used herein the term “antibody” is intended to include polyclonal and monoclonal antibodies of any isotype (IgA, IgG, IgE, IgD, IgM), or an antigen-binding portion thereof, including, but not limited to, F(ab) and Fv fragments such as scFv, single chain antibodies, chimeric antibodies, humanized antibodies, recombinant engineered antibody and a Fab expression library. Bispecific antibodies can also be immobilized on a magnetic particle.

Magnetic particles may be labeled with a binding partner such as an antibody, a protein, or a nucleic acid molecule. A first member of a specific binding pair can be associated with a magnetic particle, wherein the biomolecule to be modified comprises a moiety that binds to the member of the specific binding pair. Alternatively, the magnetic particle is coupled, e.g. to the antibody or the immunologically reactive fragment thereof, through a linker or a spacer (such as, e.g., a nucleic acid linker). Addition of spacers or linkers will allow biomolecules to be presented in a more flexible fashion, and careful chemistry can attach ligands in a specific orientation. There are numerous chemistries used for these couplings and published protocols known in the art.

Examples of members of specific binding pairs that can be attached to a magnetic particle include, but are not limited to, oligo dT (for binding to nucleic acid molecules comprising, e.g., a poly-A tract at the 3′ end); oligonucleotides having a specific nucleotide sequence (for binding to nucleic acid molecules comprising a complementary nucleotide sequence); avidin (e.g., streptavidin) (for binding to a biotinylated biomolecule); an antigen-binding polypeptide, e.g., an immunoglobulin (Ig) or epitope-binding fragment thereof (for binding to a biomolecule comprising an epitope recognized by the Ig); polynucleotide binding proteins (for binding to a polynucleotide), e.g., a transcription factor, a translation factor, and the like; Ni or Co chelate (to immobilize poly-histidine-tagged proteins); receptor-ligand systems, or other specific protein-protein interacting pairs; aptamers (e.g., nucleic acid ligands for three-dimensional molecular targets); lectins (for binding glycoproteins); lipids and phospholipids (binding to lipid-binding proteins), e.g., phosphatidyl serine and annexin V. Those skilled in the art will recognize other members of specific binding pairs that may be attached to a magnetic particle.

A biomolecule can also be coupled (covalently or non-covalently) to a magnetic particle by direct chemical conjugation or by physical association. Such methods are well known in the art. Biochemical conjugations are described in, e.g., “Bioconjugate Techniques” Greg T. Hermanson, Academic Press. Non-covalent interactions, such as ionic bonds, hydrophobic interactions, hydrogen bonds, and/or van der Waals attractions can also be used to couple a biomolecule with a magnetic particle. For example, standard non-covalent interactions used to bind biomolecules to chromatographic matrices can be used. One non-limiting example of such a non-covalent interaction that can be used to bind a biomolecule to a magnetic particle is DNA binding to silica in the presence of chaotropic salts. Those skilled in the art are aware of other such non-covalent binding and conditions for achieving the same. See, e.g., Molecular Cloning, Sambrook and Russell, Cold Spring Harbor Laboratory Press.

As used herein “magnetic particles” are used as labels for biomolecule targets in a biological sample such as, but not limited to, antibodies, DNA, polypeptides, and cells to aid in their separation from complex mixtures of a sample. Magnetic particles may be classified according to size: nanobeads which range from about <50 nm to 1 μm; and micron-sized beads that are about 1-5 μm. Furthermore, magnetic particles can be adapted for selective affinity (functionalized) for coupling or binding with a desired biomolecule target such as with a fluorescent label, antibody, nucleic acid and so forth. These magnetic particles allow a quantitative magnetic labeling of cells, thus the amount of coupled magnetic label is proportional to the amount of bound product.

As used herein “separation” includes isolation or collection accumulation of a target biological population including target cells from a surrounding fluid bulk, where the bulk is, for example, a fluidic mixture or suspension of emulsion of cells or a combination thereof, implying also concentration or enrichment of target cells relative to the surrounding bulk or a provided sample of cells (obtaining a precipitate in analogy to precipitation or centrifugation).

As used herein “depletion” with respect to separation, is the removal of a target biological population, including target cells from the bulk (obtaining a supernatant in analogy to precipitation or centrifugation).

As used herein “high qualitative” (separation, depletion) is meaning high purity, separation of target cells substantially exclusive of other cells, or comprising negligible amounts of other cells such as between about 10% and about 1% or less of the separated cells, and conversely a depletion.

As used herein “high quantitative” (separation, depletion) is meaning high recovery, separation of substantially all the target biological population target cells, or very high amount of the target cells from the sample, such as between about 80% to about 99% or more or the separated cells, and conversely a depletion.

It is noted that whenever a reference is made herein to cells attaching or sticking or adhering to a wall of a tube, or similar terms to that effect, it does not necessarily mean that the cells attach directly to the wall, but rather, that they also connect or link or are attracted indirectly to the wall such as by chains of cells or groups of cells.

As used herein an “electromagnet” is a type of magnet in which the magnetic field is produced by an electric current. The magnetic field disappears when the current is turned off. Electromagnets usually consist of wire wound into a coil. A current through the wire creates a magnetic field which is concentrated in the hole in the center of the coil. The wire turns are often wound around a magnetic core made from a ferromagnetic or ferrimagnetic material such as iron; the magnetic core concentrates the magnetic flux and makes a more powerful magnet.

As used herein a “permanent magnet” is a magnet that is permanent, in contrast to an electromagnet, which only behaves like a magnet when an electric current is flowing through it. Permanent magnets are made out of substances like magnetite (Fe₃O₄), the most magnetic naturally occurring mineral, or neodymium alloy, a popular magnetic alloy.

As used herein “magnet array” is one or more magnets. The one or more magnets can be permanent magnets or electromagnets. One or more permanent magnets may be in a linear array, in different sizes, different strengths, configured in opposite pole directions perpendicular to the axis of the linear array or configured with 90° rotations to one another in a plane perpendicular to the axis of the linear array or any other angle. Any number of magnets in the array may be physically held together or adhesively held together. Permanent magnets may be of a material selected from iron, neodymium, samarium-cobalt, or alnico.

As used herein “fluidic pathway” may refer to a tube or other open-ended structure having a flow path moving therethrough in at least one direction, or in embodiments, multiple directions. Alternatively, “fluidic pathway” may refer to a container or holding structure (e.g., anything that can hold fluid such as cells, media, etc.) for the biological sample and/or magnetic particles as described herein. It should be understood that that the term “fluidic pathway” is interchangeable by definition in this regard, and may be applied as such to all embodiments of the magnetic separation system as further described herein.

The present disclosure relates to devices, methods and systems for magnetically separating and collecting a desired target biological population in a biological sample through positive or negative selection. As presented herein, a magnetic field is produced that is substantially adjacent to a biological sample containing a desired magnetized target biological population. The magnetic field can be switched “ON” or “OFF” in an automatic manner such to provide a magnetic field of a desired strength, continuous time duration, intermitted duration, pulsation duration and combinations thereof. This may be achieved by a swivel component or otherwise translatable component that allows for translation of the source of the magnetic field or magnetic field gradient towards and away from the biological sample and/or magnetic particles.

The isolation of a target biological population such as target cells from a biological sample is achieved herein by binding the target biological population such as target cells to magnetic particles and subjecting the bound target cells to the magnetic field as the biological sample and magnetic particles flow through or are contained within the fluidic pathway. Larger sized particles (on the magnitude of micrometers) have been used in prior magnetic separation systems. However, it has been found that the use of smaller sized particles (on the magnitude of nanometers) is preferred in the isolation of target cells as they have less impact on a patient following implantation. Prior systems have presented difficulties in isolating nanosized magnetic particles due to their very small size, and the efficiency of isolating such nanosized magnetic particles is further challenged from space constraints and automated requirements of magnetic separation systems. For example, prior automated systems separate nanobeads through the use of columns filled with paramagnetic beads. The fluid and cells bound to the nanobeads are flowed through these columns and directly contact the paramagnetic beads. This system is not ideal as it can be difficult to harvest the cells once they are magnetically drawn to the paramagnetic beads. As another example, prior systems include large manual quadrupole technologies. The drawback to these systems is the necessity of manual interaction for operation. Furthermore, the fluid capacities of such systems are relatively small, and a static period of processing for multiple batches is required over long periods of time to accommodate the volumes required for clinical use. The invention as presented herein thus overcomes the difficulties of efficient separation or isolation of nanosized magnetic particles in a manner that can be achieved through automated procedures, and can yield sufficient volumes necessary for clinical use. More particularly, the invention as presented herein utilizes a novel magnet design and fluidic pathway to achieve these advances in the art. The magnet design includes numerous small magnets arranged in a Halbach array, and the fluidic pathway is designed to allow for clinical volumes to be processed in relatively short durations, which minimizes the impact on the cells, and reduces the overall processing time. Furthermore, the invention as presented herein provides an effective approach to the release of larger magnetic particles that have been captured or gathered via the magnetic field of the magnetic separation system. The fluidic pathway is further designed to achieve a low shear stress necessary to enable efficient capture of nanosized magnetic particles. The embodiments of the automated magnetic separation system that achieve these improvements will now be described in greater detail.

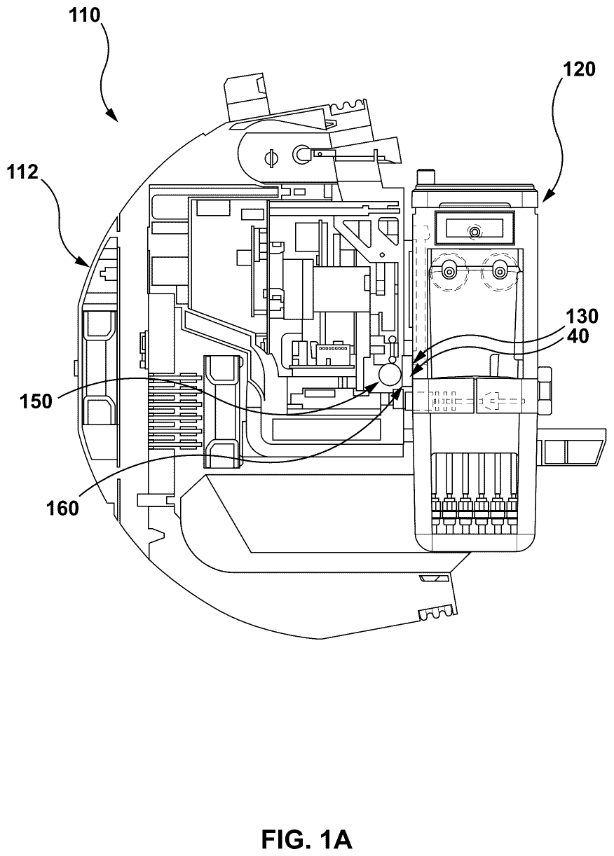

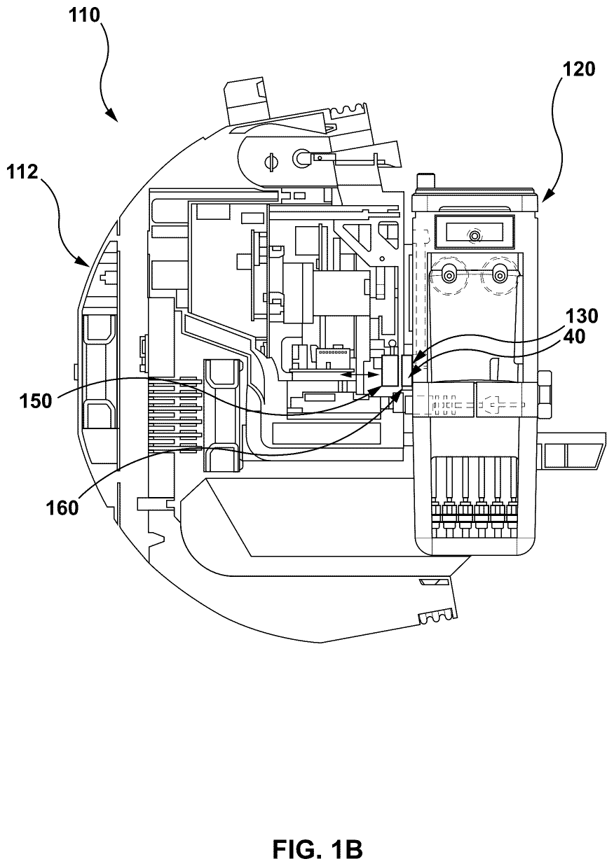

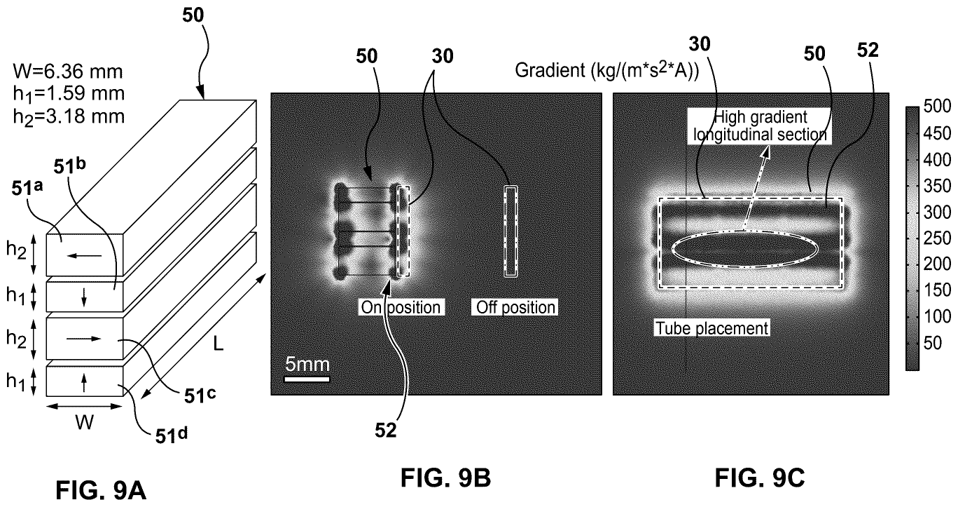

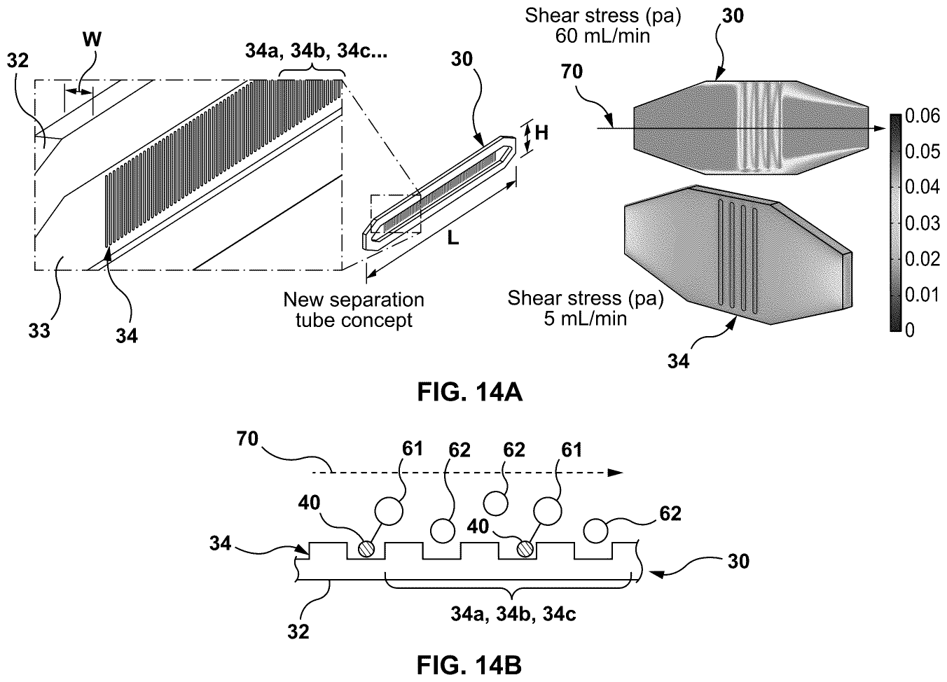

An exemplary embodiment of a magnetic separation system 110 as shown in FIGS. 1A and 1B comprises a housing 112 for receiving or securing a cell engineering cassette 120 thereto. Within or adjacent to the cell engineering cassette 120, and disposed between the housing 112 and the cell engineering cassette 120, is a fluidic pathway 130 configured for the flow of a biological sample 160 and/or magnetic particles 40 therethrough. In the example of FIG. 1A, the fluidic pathway 130 is configured as a tube or cylinder disposed adjacent to the cell engineering cassette 120. Adjacent to, or in proximity with, the fluidic pathway 130 and the biological sample 160 is an array of magnets 150 configured to generate a magnetic field gradient or magnetic field 52 for the separation and collection of a target population or a non-target population, as will be further described herein with respect to FIGS. 9A-9C. In the example of FIG. 1B, the fluidic pathway 130 is disposed within or adjacent to the cell engineering cassette 120, between the cell engineering cassette 120 and the housing 112. The fluidic pathway 130 is configured as a parallelepiped as further described herein with reference to FIG. 14. In the example of FIG. 1B, the array of magnets 150 may translate or swivel or translate toward and away from the fluidic pathway 130 and the biological sample 160 to facilitate the separation and collection of a target population or a non-target population, as illustrated by the arrows.

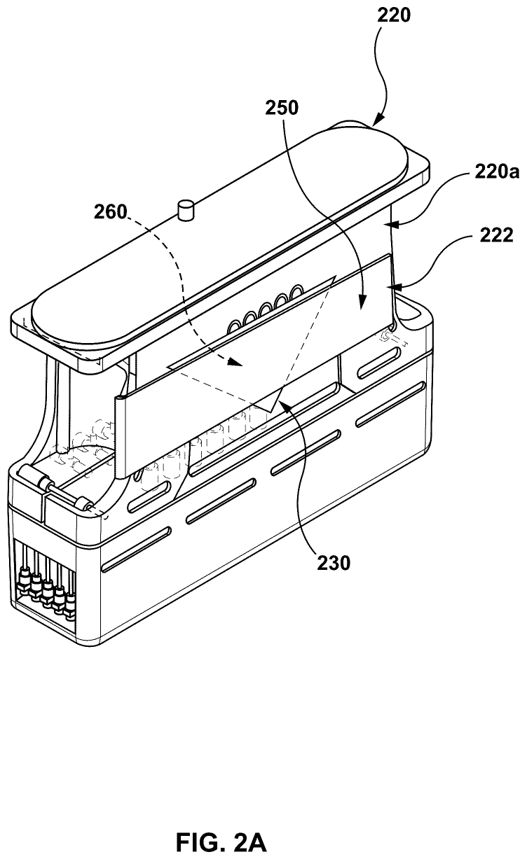

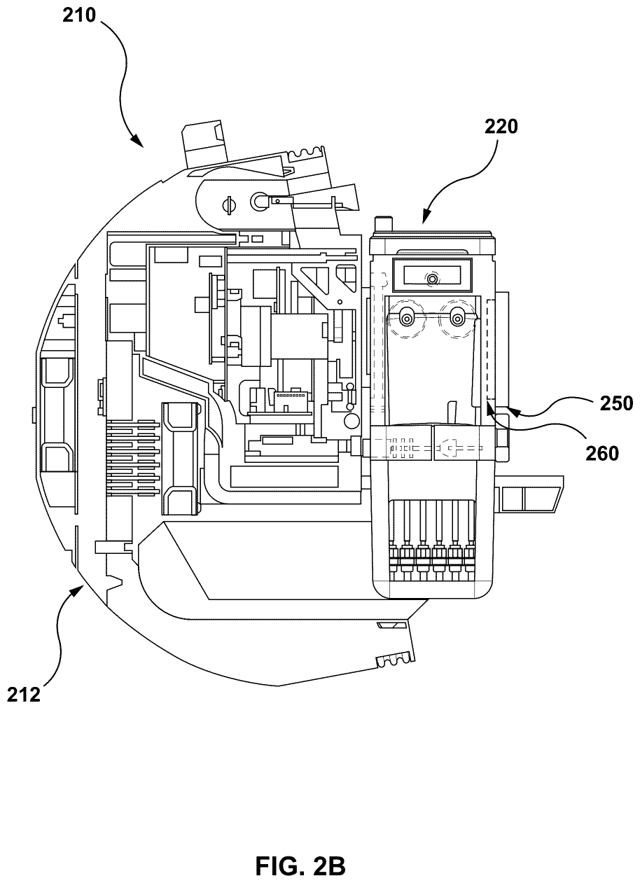

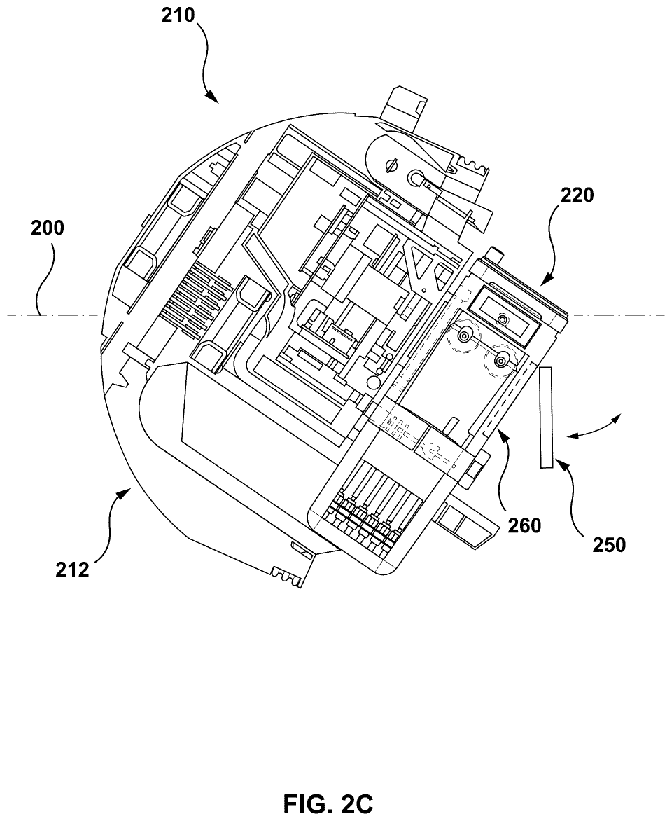

Another exemplary embodiment of the magnetic separation system 210 as shown in FIGS. 2A-2C comprises the array of magnets 250 being disposed on a satellite chamber 222 of the cell engineering cassette 220. FIG. 2A illustrates an embodiment of the cell engineering cassette 220 with the array of magnets 250 disposed adjacent to a sidewall 220a of the cell engineering cassette 220 on the satellite chamber 222. The fluidic pathway 230 which the biological sample 260 and/or magnetic particles 40 flows therethrough or is disposed therein is also disposed on or adjacent to the satellite chamber 222. The biological sample 260 and/or magnetic particles 40 is contained in or flowed through the fluidic pathway 230 that is disposed on or adjacent to the sidewall 220a of the cell engineering cassette 220. FIG. 2B illustrates an example of the cell engineering cassette 220 disposed on or within the housing 212 of the magnetic separation system 210. Here, the array of magnets 250 are engaged with the biological sample 260, such that the magnetic field or magnetic field gradient 52 is applied to the biological sample 260 for the separation and collection of the target biological population or non-target biological population, or the first subpopulation or second subpopulation, as further described herein. As shown in FIG. 2C, the array of magnets 250 is configured to swing or translate away from the cell engineering cassette 220 (as illustrated by the arrows in FIG. 2C), either by tilting or rotating the housing 212 off the housing's horizontal axis 200, by manually swiveling the array of magnets 250 (e.g., through end-user interaction), or by automatically swiveling the array of magnets 250 (e.g., via a servomotor or other actuator mechanically and/or electrically engaged with the magnetic separation system 210).

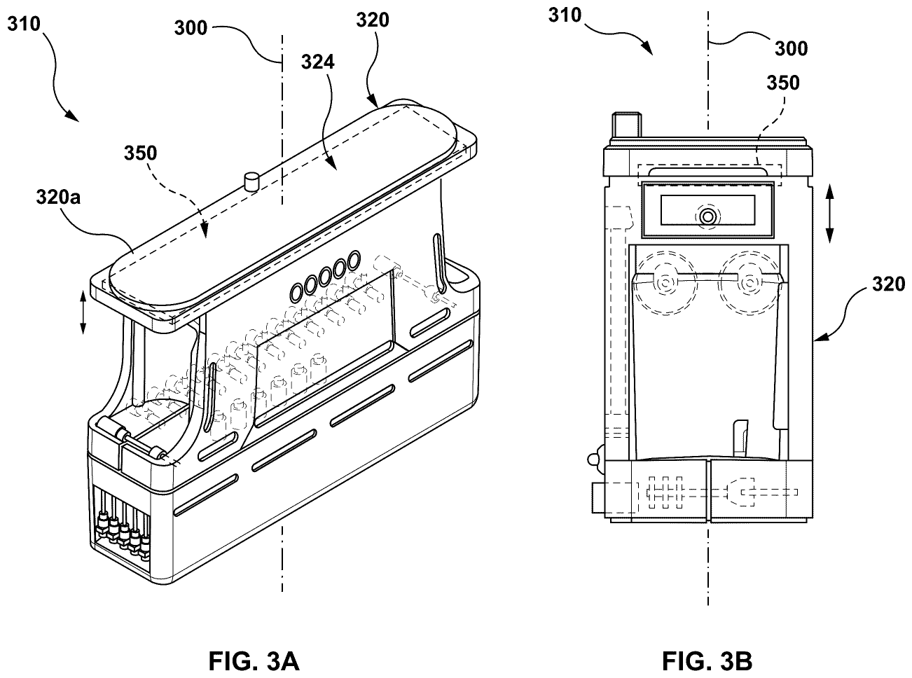

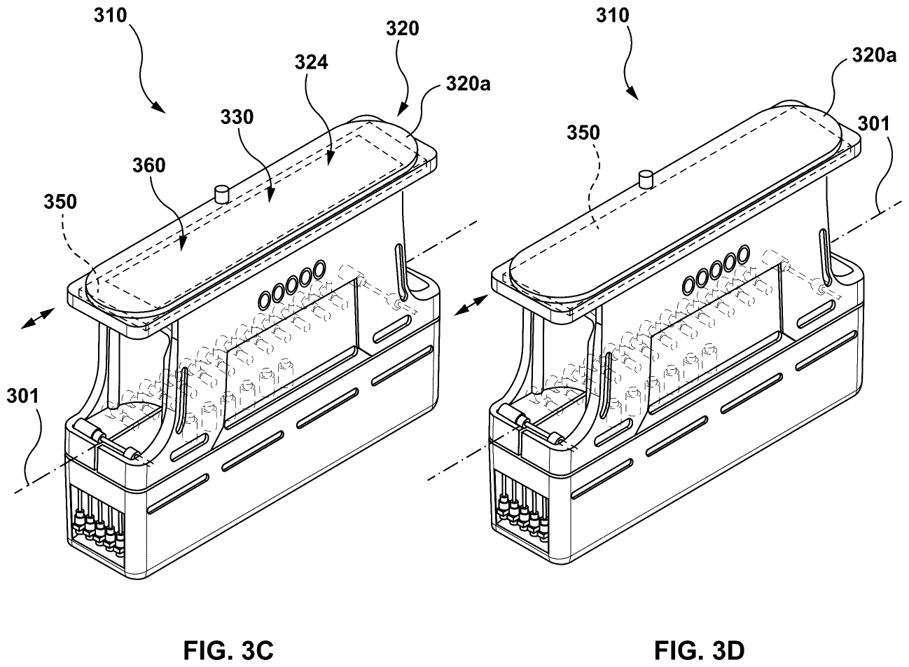

Another exemplary embodiment of the cell engineering cassette 320 for use with the magnetic separation system 310 as shown in FIGS. 3A-3D comprises the array of magnets 350 disposed below or adjacent to a proliferation chamber 324 on a top surface 320a of the cell engineering cassette 320. The fluidic pathway 330 which the biological sample 360 flows therethrough contains the proliferation chamber 324 as a subcomponent of the fluidic pathway 330. As shown in FIG. 3A and FIG. 3B, the proliferation chamber 324 may be configured to be raised or lowered along a vertical axis 300 (the movement as indicated by the arrows) of the cell engineering cassette 320 to adjust the distance between the array of magnets 350, and the fluidic pathway 330 in which the biological sample 360 and/or magnetic particles 40 flows therethrough or is otherwise contained therein. Alternatively, the array of magnets 350 may be configured to be raised or lowered along the vertical axis 300 of the cell engineering cassette 320 to adjust the distance between the array of magnets 350 and the fluidic pathway 330 in which the biological sample 360 and/or magnetic particles 40 flows therethrough or is otherwise contained therein. Adjusting the distance between the array of magnets 350 and the fluidic pathway 330 in this manner allows for application or de-application of the magnetic field or magnetic field gradient 52 (as further described with respect to FIGS. 9A-9C below) to the biological sample 360 and/or magnetic particles 40 for the separation and collection of the target biological population or non-target biological population, or the first subpopulation or second subpopulation, as further described herein. Alternatively, as shown in FIG. 3C and FIG. 3D, the proliferation chamber 324 may slide along a horizontal axis 301 of the cell engineering cassette 320 (indicated by the arrows) to adjust the distance between the array of magnets 350 and the fluidic pathway 330 which the biological sample 360 and/or magnetic particles 40 flows therethrough or is otherwise contained therein. In another embodiment, the array of magnets 350 may slide along the horizontal axis 301 of the cell engineering cassette 320 to adjust the distance between the array of magnets 350 and the fluidic pathway 330 which the biological sample 360 and/or magnetic particles 40 flows therethrough or is otherwise contained therein. Adjusting the distance between the array of magnets 350 and the fluidic pathway 330 in this manner allows for application or de-application of the magnetic field or magnetic field gradient 52 (as further described with respect to FIGS. 9A-9C below) to the biological sample 360 and/or magnetic particles 40 for the separation and collection of the target biological population or non-target biological population, or the first subpopulation or second subpopulation, as further described herein.

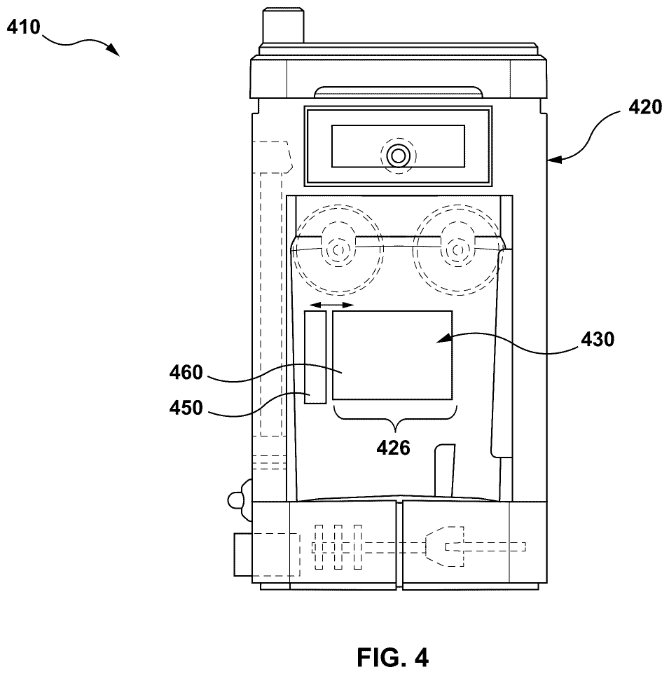

Another exemplary embodiment of the cell engineering cassette 420 for use with the magnetic separation system 410 as shown in FIG. 4 comprises the array of magnets 450 being disposed in, beside, or on an external surface of a crossflow reservoir 426 of the cell engineering cassette 420. The fluidic pathway 430 which the biological sample 460 and/or magnetic particles 40 flows therethrough or is otherwise contained therein may be disposed adjacent to the array of magnets 450 in an orientation perpendicular to the array of magnets 450. As shown in FIG. 4, the array of magnets 450 may be moved towards and away from the fluidic pathway 430 (as indicated by the arrows) to adjust the distance between the array of magnets 450 and the fluidic pathway 430 which the biological sample 460 and/or magnetic particles 40 flows therethrough or is otherwise contained therein. Adjusting the distance between the array of magnets 450 and the fluidic pathway 430 in this manner allows for application or de-application of the magnetic field or magnetic field gradient 52 (as further described with respect to FIGS. 9A-9C below) to the biological sample 460 and/or magnetic particles 40 for the separation and collection of the target biological population or non-target biological population, or first subpopulation or second subpopulation, as further described herein.

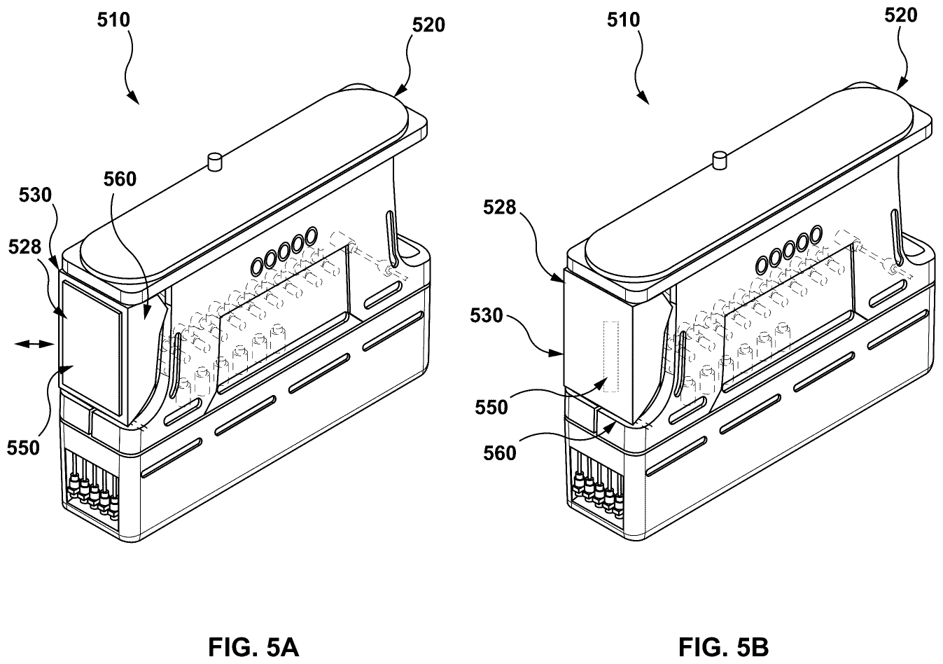

Another exemplary embodiment of the cell engineering cassette 520 for use with the magnetic separation system 510 as shown in FIG. 5A and FIG. 5B comprises the array of magnets 550 being disposed on or within a container module 528 of the cell engineering cassette 520. In the example of FIG. 5A, the array of magnets 550 may be disposed on an external surface of the container module 528 within the fluidic pathway 530 which the biological sample 560 and/or magnetic particles 40 flows therethrough or is otherwise contained therein. The array of magnets 550 may translate, pivot, or otherwise be removed from the container module 528 to allow for application or de-application of the magnetic field or magnetic field gradient 52 (as further described with respect to FIGS. 9A-9C below) to the biological sample 560 and/or magnetic particles 40 for the separation and collection of the target biological population or non-target biological population, or first subpopulation or second subpopulation as further described herein. Alternatively, as shown in FIG. 5B, the array of magnets 550 may be disposed internally in the container module 528 adjacent to the fluidic pathway 530 which the biological sample 560 and/or magnetic particles 40 flows therethrough or is otherwise contained therein. The array of magnets 550 may translate, pivot, or otherwise be removed from the container module 528 to allow for application or de-application of the magnetic field or magnetic field gradient 52 (as further described with respect to FIGS. 9A-9C below) to the biological sample 560 and/or magnetic particles 40 for the separation and collection of the target biological population or non-target biological population, or first subpopulation or second subpopulation as further described herein.

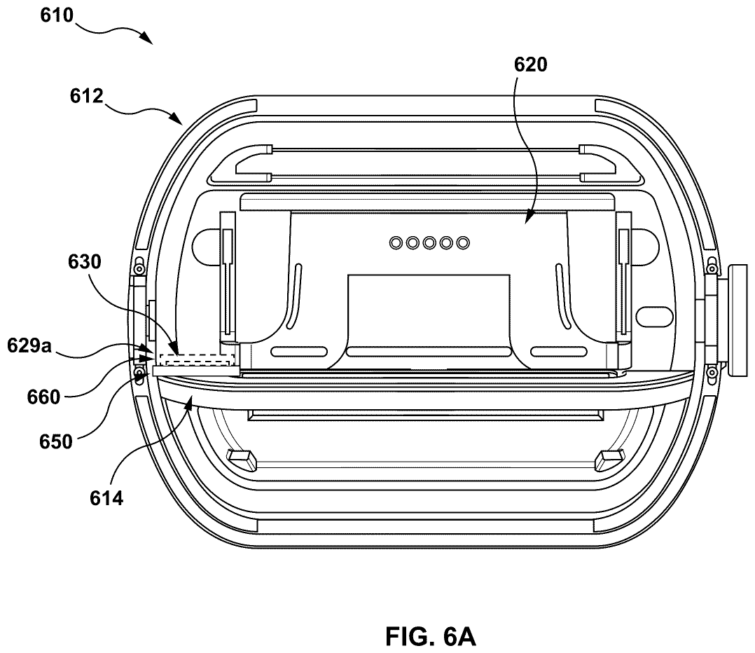

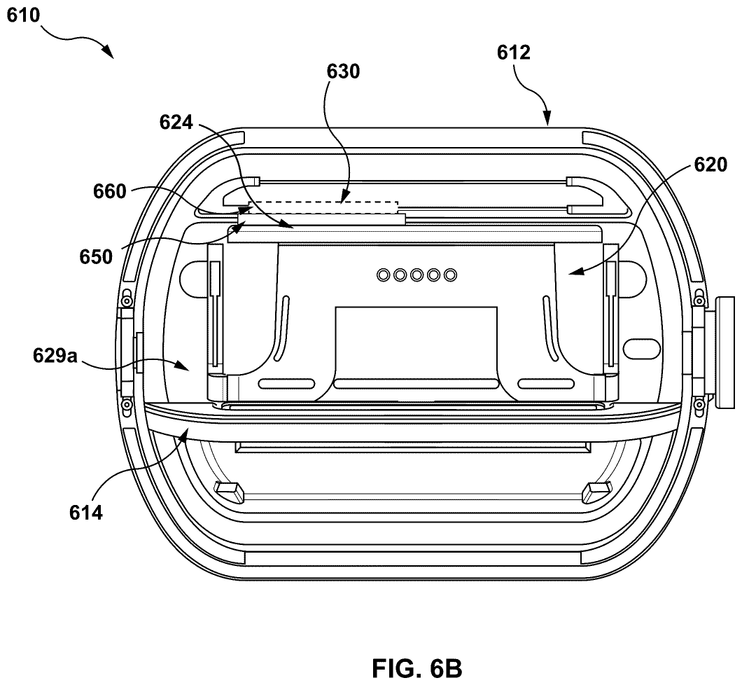

Another exemplary embodiment of the magnetic separation system 610 as shown in FIG. 6A and FIG. 6B illustrates the region of the warm zone 629a within the housing 612 that contains the magnet array 650, the warm zone 629a which may be suitably maintained at approximately 30-45° C., e.g. about 37° C. In the example of FIG. 6A, the magnetic separation system 610 comprises the array of magnets 650 being disposed adjacent to the warm zone 629a within the housing 612 of the system 610. The warm zone 629a is formed in an upper portion above a movable locking arm or thermal barrier 614, such that the upper portion of the movable locking arm or thermal barrier 614 is located within the warm zone 629a, the thermal barrier 614 which is configured to secure the cell engineering cassette 620 within the housing 612 after the cell engineering cassette 620 is installed into the housing 612. The fluidic pathway 630 is also disposed on or adjacent to the warm zone 629a on the thermal barrier 614, and the biological sample 660 and/or magnetic particles 40 is contained within or flowed through the fluidic pathway 630 adjacent to the array of magnets 650 on or adjacent to the warm zone 629a on the thermal barrier 614. In the example of FIG. 6B, the fluidic pathway 630 is disposed on a proliferation chamber 624 of the cell engineering cassette 620, separate from the thermal barrier 614 which is disposed on or integral with the movable locking arm or thermal barrier 614. The array of magnets 650 is also disposed adjacent to or on the proliferation chamber 624 of the cell engineering cassette 620, in between the cell engineering cassette 620 and the fluidic pathway 630. The biological sample 660 and/or magnetic particles 40 is flowed through or otherwise contained within the fluidic pathway 630 and exposed to the magnetic field or magnetic field gradient 52 emitted from the array of magnets 650 for facilitating the separation and or collection of the target or non-target population, or first subpopulation or second subpopulation as further described herein. In embodiments, the warm zone 629a of FIG. 6B may instead contain the proliferation chamber 624, some or all of the fluidic pathway 630, array of magnets 650, and/or biological population 660. In embodiments, the warm zone 629a may include the entire upper portion of the cell engineering cassette 620, i.e., the portions that are above the thermal barrier 614, such as the proliferation chamber 624, some or all of the fluidic pathway 630, the array of magnets 650, and/or the biological sample 660.

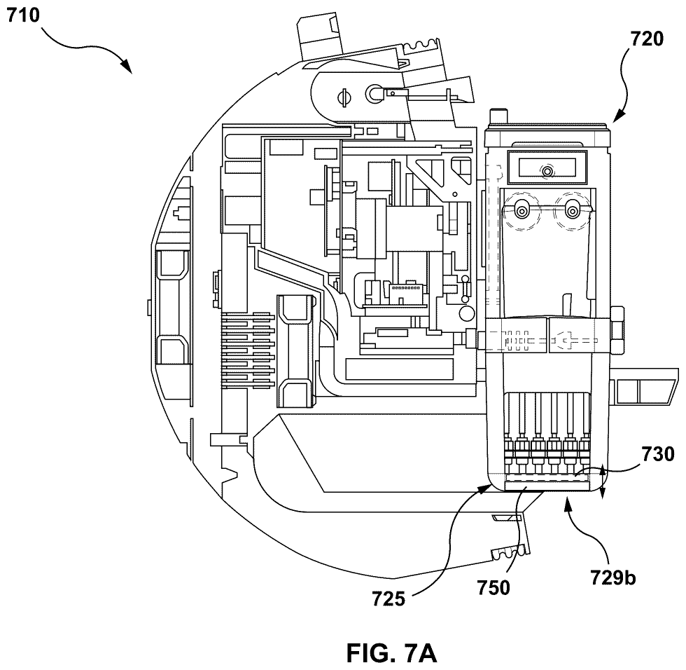

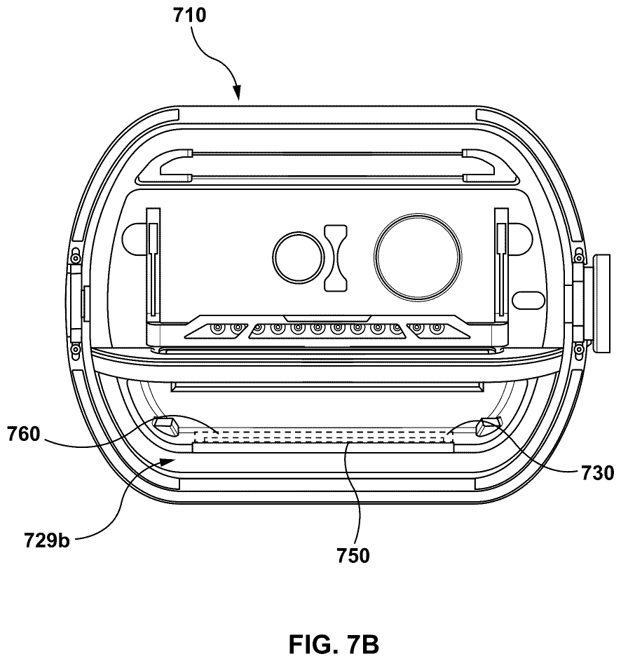

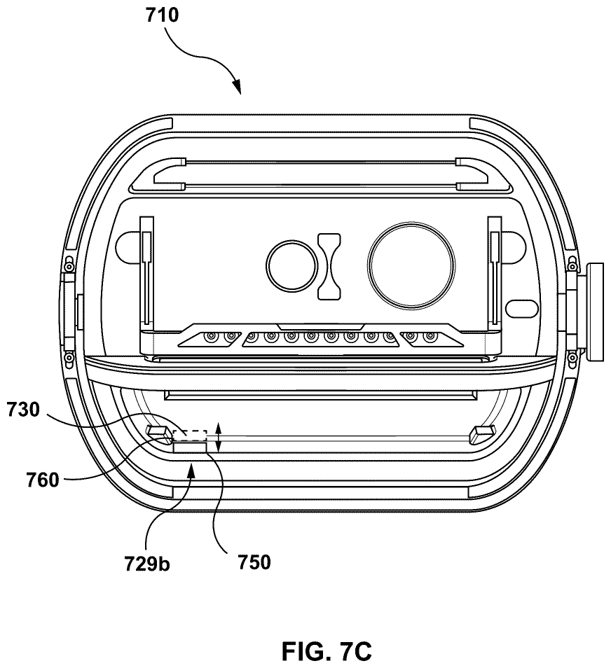

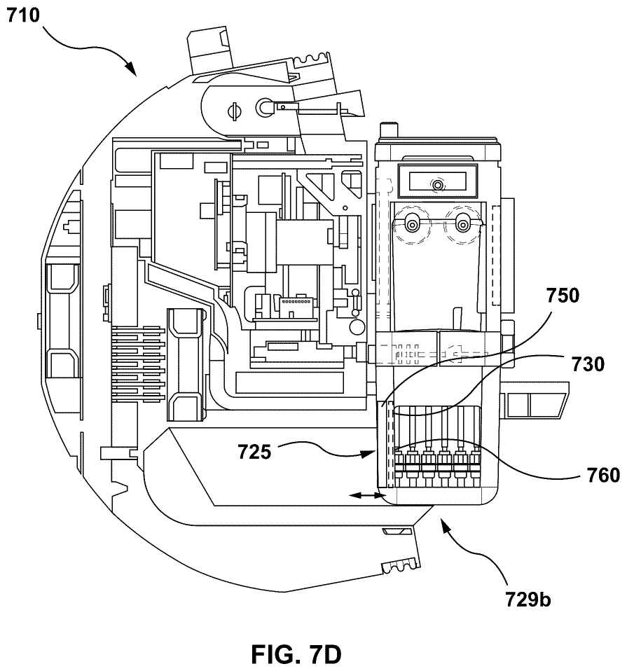

Another exemplary embodiment of the magnetic separation system 710 as shown in FIGS. 7A-7D comprises the array of magnets 750 being disposed in various locations in the cold zone 729b of the magnetic separation system 710 within the housing 712 or the cell engineering cassette 720. In the example of FIG. 7A, the array of magnets 750 is disposed on or within a fluid reservoir 725 located on a bottom surface of the cell engineering cassette 720. In embodiments, the fluid reservoir 725 may be adjacent to or integral with a cold zone 729b for the low temperature storage, e.g. approximately 2-15° C., or about 8° C., or processing of a biological sample 760. The array of magnets 750 may be disposed on the bottom surface of the cell engineering cassette 720, and the fluidic pathway 730 may be disposed between the array of magnets 750 and the bottom surface of the cell engineering cassette 720, adjacent to or integral with the cold zone 729b. In the example of FIG. 7B, the array of magnets 750 is disposed on or within a bottom surface of the housing 712. The fluidic pathway 730 may be disposed adjacent to the array of magnets 750. The fluid reservoir 725 may be adjacent to or integral with a cold zone 729b for the low temperature storage or processing of a biological sample 760. In the example of FIG. 7C, the array of magnets 750 is disposed on or within a bottom edge or bottom surface of the housing 712. The fluidic pathway 730 may be disposed adjacent to the array of magnets 750. The fluid reservoir 725 may be adjacent to or integral with a cold zone 729b for the low temperature storage or processing of a biological sample 760. The array of magnets 750 may further be translatable toward or away from the fluidic pathway 730 to adjust the distance between the array of magnets 750, and the fluidic pathway 730 which the biological sample 760 and/or magnetic particles 40 flows therethrough or is otherwise contained therein. Adjusting the distance between the array of magnets 750 and the fluidic pathway 730 in this manner allows for application or de-application of the magnetic field or magnetic field gradient 52 (as further described with respect to FIGS. 9A-9C below) to the biological sample 760 and/or magnetic particles 40 for the separation and collection of the target biological population or non-target biological population, or the first subpopulation or second subpopulation, as further described herein. In the example of FIG. 7D, the array of magnets 750 is disposed on or within a fluid reservoir 725 located on a bottom side surface, a back wall, or a back vertical surface of the cell engineering cassette 720. The fluidic pathway 730 may be disposed adjacent to the array of magnets 750. The fluid reservoir 725 may be adjacent to or integral with a cold zone 729b for the low temperature storage or processing of a biological sample 760. The array of magnets 750 may further be translatable toward or away from the fluidic pathway 730 to adjust the distance between the array of magnets 750, and the fluidic pathway 730 which the biological sample 760 and/or magnetic particles 40 flows therethrough or is otherwise contained therein. Adjusting the distance between the array of magnets 750 and the fluidic pathway 730 in this manner allows for application or de-application of the magnetic field or magnetic field gradient 52 (as further described with respect to FIGS. 9A-9C below) to the biological sample 760 and/or magnetic particles 40 for the separation and collection of the target biological population or non-target biological population, or the first subpopulation or second subpopulation, as further described herein.

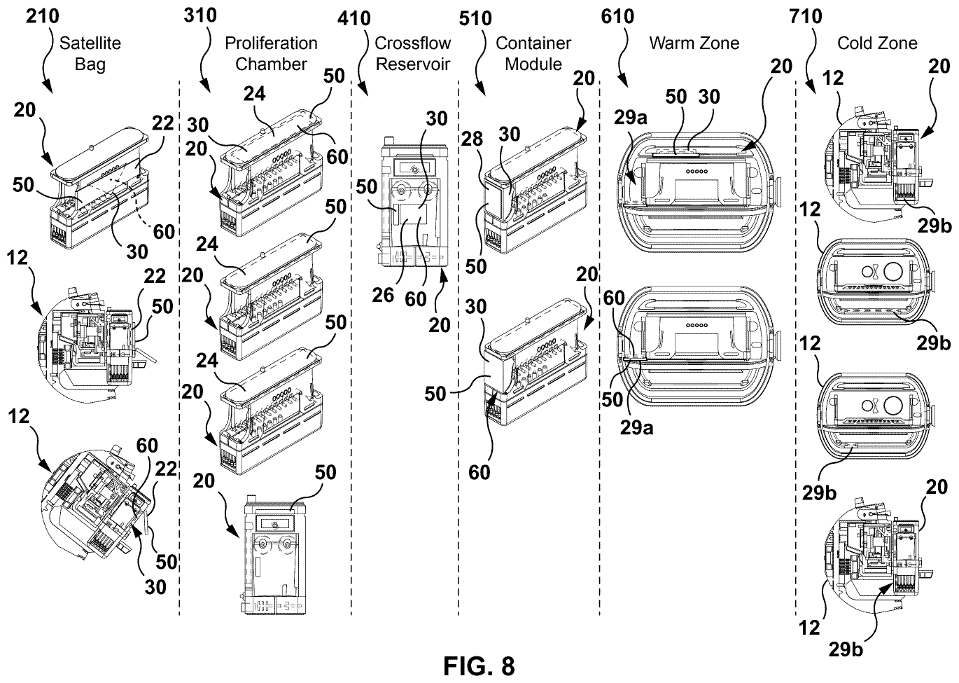

FIG. 8 depicts some of the embodiments of the magnetic separation system 210/310/410/510/610/710 previously described herein. As should be understood by the present disclosure, the various components described herein with respect to FIGS. 1A-7D may be interchangeable between the various magnetic separation systems 110/210/310/410/510/610/710 previously described, and should not be interpreted as being limited to the embodiments illustrated across FIGS. 1A-7D. In general, the magnetic separations systems 110/210/310/410/510/610/710 may comprise some or all embodiments of the magnetic separation system components and features, including, but not limited to, housing 12, cell engineering cassette 20, satellite chamber 20, proliferation chamber 24, crossflow reservoir 26, container module 28, warm zone 29a, cold zone 29b, magnetic particles 40, array of magnets 50, biological sample 60, and fluidic pathway 30, among other components. FIG. 8 serves to illustrate the modular capabilities of the components of the magnetic separation systems 110/210/310/410/510/610/710 previously described, as these magnetic separation systems 110/210/310/410/510/610/710 are all configured to contain, interact with, or otherwise operate with embodiments of the array of magnets 50, magnetic field or field gradient 52, swivel or translation component 54, magnetic particles 40, and fluidic pathway 30 configured to achieve the inventive improvements previously described above with respect to prior magnetic separation systems.

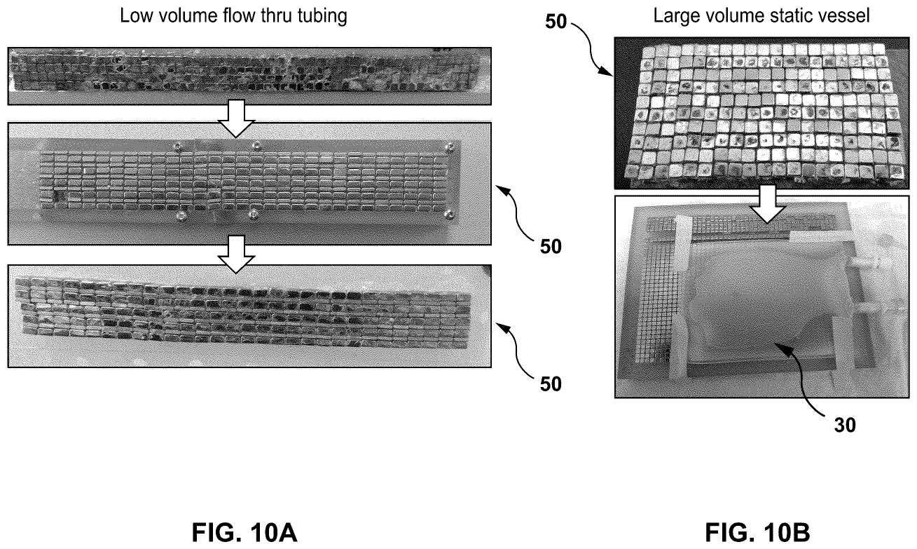

FIGS. 9A-10B generally depict the array of magnets 50 used in the various embodiments of the magnetic separation system 110/210/310/410/510/610/710 previously described above. As shown in FIG. 9A, the array of magnets 50 used in the various embodiments of the magnetic separation system 110/210/310/410/510/610/710 may be configured in a Halbach array, such that each magnet 51 in the array of magnets is arranged in a spatially rotating pattern of magnetization. In other words, the array of magnets 50 are arranged such that the magnetic field of each magnet 51a, 51b, 51c, 51d is oriented to face a direction 90° from the direction of an adjacent magnet 51a, 51b, 51c, 51d, as demonstrated by the arrows shown on each magnet 51a, 51b, 51c, 51d in FIG. 9A. By arranging each magnet 51a, 51b, 51c, 51d of the array of magnets 50 in this configuration, the magnetic field or magnetic field gradient 52 emitted from the array of magnets 50 is augmented on one side of the array of magnets 50, while the magnetic field gradient 52 emitted from the opposite side of the array of magnets 50 is significantly reduced or cancelled entirely as shown in FIG. 9B. In embodiments, each magnet 51a, 51b, 51c, 51d of the array of magnets 50 may comprise a width of approximately 3-10 mm, and preferably about 6.36 mm, and a length of 10-75 mm, and preferably about 25-50 mm. When arranged as the array of magnets 50, a first pair of alternating magnets 51b, 51d may comprise a height H1 of approximately 0.5-6 mm, and preferably about 1.59 mm, while a second pair of alternating magnets 51a, 51c may comprise a height H2 of approximately 2-5 mm, and preferably about 3.18 mm, as exemplified in FIG. 9A.

FIG. 9B depicts the array of magnets 50 in the ON and OFF positions, such that the magnetic field or magnetic field gradient 52 emitted from the array of magnets 50 is engaged with the fluidic pathway 30 when in the ON position, and disengaged from the fluidic pathway 30 when in the OFF position. In embodiments, the array of magnets 50 may be switched between the ON and OFF positions by translating or swiveling the array of magnets 50 toward or away from the fluidic pathway 30, respectively. In embodiments, the array of magnets 50 may be electromagnets, and may be switched between the ON and OFF positions by the application or de-application of an electric current to the array of magnets 50. FIG. 9C depicts the array of magnets 50 in the ON position (i.e., engaging the fluidic pathway 30 with the magnetic field or magnetic field gradient 52) from a side elevational view, such that the high gradient of the magnetic field or magnetic field gradient 52 emitted from the array of magnets 50 is highlighted in the areas where the magnetic field 52 is concentrated due to the Halbach array formation of the array of magnets 50. In embodiments, the array of magnets 50 may be switched between the ON and OFF positions by translating or adding a magnetic shield or barrier (not shown) between the array of magnets 50 and the fluidic pathway 30, such that the magnetic shield or barrier blocks or disrupts the magnetic field or magnetic field gradient 52 interaction with the fluidic pathway 30.

FIGS. 10a and 10b depict examples of the array of magnets 50 used in the various embodiments of the magnetic separation system 110/210/310/410/510/610/710 previously described above. In some embodiments, the magnetic separation system 110/210/310/410/510/610/710 may utilize a low volume flow thru tubing, such that the array of magnets 50 is arranged in an oblong manner to facilitate magnetic separation over the length of the fluidic pathway while the biological population 60 being separated is under flow. When configured in this manner, the array of magnets 50 may be disposed along the length of the fluidic pathway 30 for application of the magnetic field or magnetic field gradient 52 thereto (FIG. 10A). In other embodiments, the magnetic separation system 110/210/310/410/510/610/710 may utilize a large volume static vessel, such that the array of magnets 50 is configured in a larger sized array having approximately 1000-2000 total individual magnets 51 (FIG. 10B). When configured in this manner, the array of magnets 50 may be disposed adjacent to a larger fluidic pathway 30 configured for containment of the biological population 60 and/or magnetic particles 40 therein.

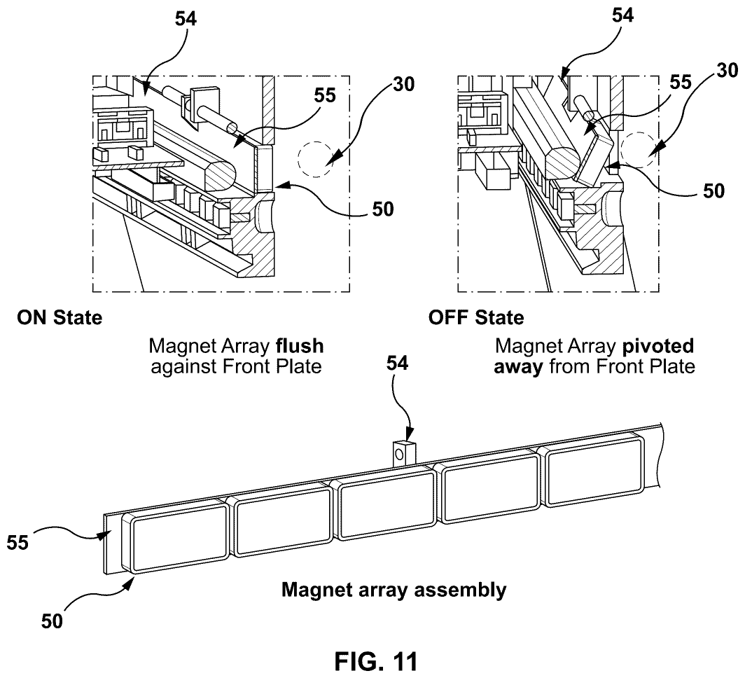

FIG. 11 depicts an embodiment of a translatable component or swivel 54 having a swivel plate or face 55 configured to receive or adhere the array of magnets 50 thereto. The array of magnets 50 may be disposed substantially along the swivel plate or face 55 and adhered to the swivel plate or face 55 via adhesives, bolts, screws, rivets, or any other suitable means for securing the array of magnets 50. In embodiments, the swivel 54 is configured to translate the array of magnets 50 towards and away from the fluidic pathway 30 in the ON and OFF positions as previously described herein, for engaging and disengaging the magnetic field or magnetic field gradient 52 to the biological sample 60 flowing through the fluidic pathway 30. For example, in the ON position, the swivel 54 is arranged such that the array of magnets 50 sits flush against the fluidic pathway 30 of the cell engineering cassette 20 (not shown). In the OFF position, the swivel 54 is arranged such that the array of magnets 50 is pivoted away from the fluidic pathway 30 of the cell engineering cassette 20 (not shown).

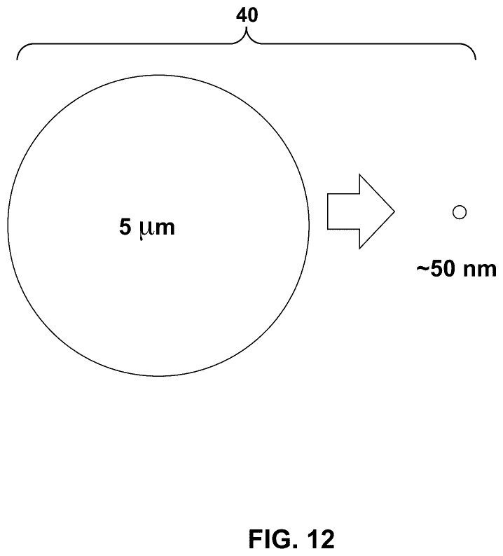

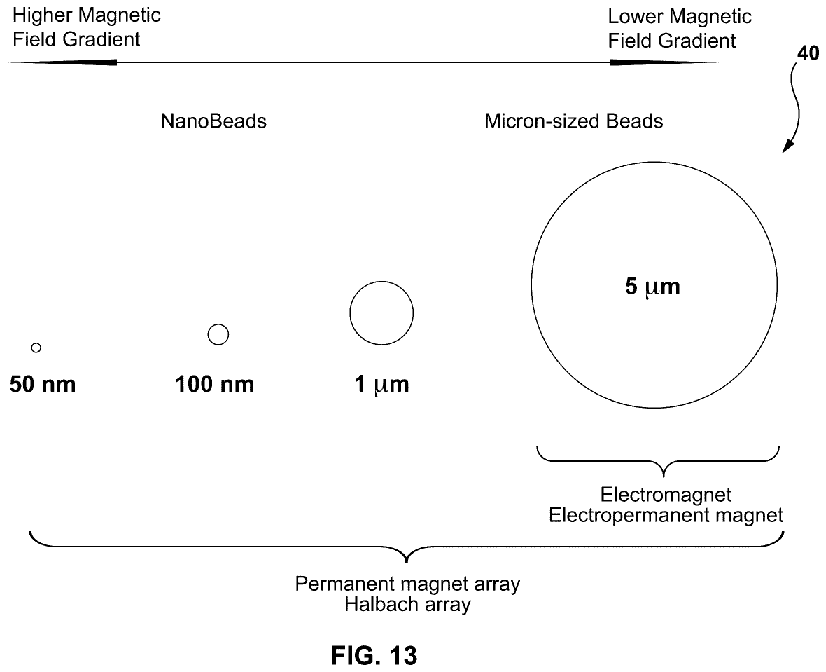

FIGS. 12 and 13 depict embodiments of the magnetic particles 40 for use with the magnetic separation system 110/210/310/410/510/610/710 described herein. The magnetic particles are spherical particles and may comprise a diameter between 50 nanometers to 5 micrometers. The magnetic particles 40 are sized to flow through the fluidic pathway 30 in accordance with the embodiments described herein.

FIGS. 14A and 14B depict an embodiment of the fluidic pathway 30 for use with the magnetic separation system 110/210/310/410/510/610/710 described herein. In the embodiment of FIG. 14A, the fluidic pathway 30 may be configured as a parallelepiped having tapered edges towards the ends of its length. In embodiments, the fluidic pathway may have a height H of approximately 5-44 mm, and preferably 16 mm, a width W of approximately 0.5-6 mm, and preferably 2 mm, and a length L of approximately 50-520 mm, and preferably 180 mm. The fluidic pathway may comprise a housing 32 having sidewalls 33 configured with a plurality of entrapment features 34 formed thereon along the length L. In embodiments, the fluidic pathway 30 may have a wall thickness of approximately 0.5-3 mm for each sidewall 33 of the housing 32, and preferably a thickness of 1.4 mm. The entrapment features 34 disposed along the sidewalls or the length of the fluidic pathway may be configured such that each individual entrapment feature 34a, 34b, 34c, etc. may have a height and/or a width of about 0.05-1 mm, and preferably 0.3 mm. Each individual entrapment feature 34a, 34b, 34c, etc. may be spaced apart approximately 1 mm to 5 mm from each other, or from an adjacent entrapment feature 34a, 34b, 34c, etc. The cross-sectional area of the entrapment features 34 may be formed as square, rectangular, triangular, semicircular, or semioval formations in the sidewall 33 of the fluidic pathway 30 along the length L of the fluidic pathway 30, or along a flow path 70 running therethrough. The entrapment features 34 may further be formed from a rigid or flexible material. In the embodiment shown in FIG. 14B, the entrapment features 34 are formed substantially perpendicular to the flow path 70 of the fluidic pathway 30 (i.e., along the length L). In embodiments, the entrapment features 34 may be formed at an angle substantially between 0°-90° to the flow path 70 of the fluidic pathway 30. In embodiments, the entrapment features 34 may be formed at an angle of approximately 45° to the flow path 70 of the fluidic pathway 30. The entrapment features 34 are configured to generate a shear stress between 0 to 1 Pascals, and preferably 0 to 0.06 Pascals, along the flow path 70 running through the fluidic pathway 30 at a flow rate between 1 mL/min to 100 mL/min.

The flow path 70 of the fluidic pathway 30 may be in the form of a single direction flow, i.e., in one direction along the length L of the fluidic pathway 30. In embodiments, the flow path 70 of the fluidic pathway 30 may be in the form of a multi-directional flow, i.e., in more than one direction along the length L of the fluidic pathway 30. In embodiments, the magnetic separation system 110/210/310/410/510/610/710 may provide the flexibility to use at least one, or multiple passes along the flow path 70 of the fluidic pathway 30. Additional passes (i.e., greater than one) results in an increased, or greater yield of collected target biological population or non-target biological population, or first subpopulation or second subpopulation, as further described herein.

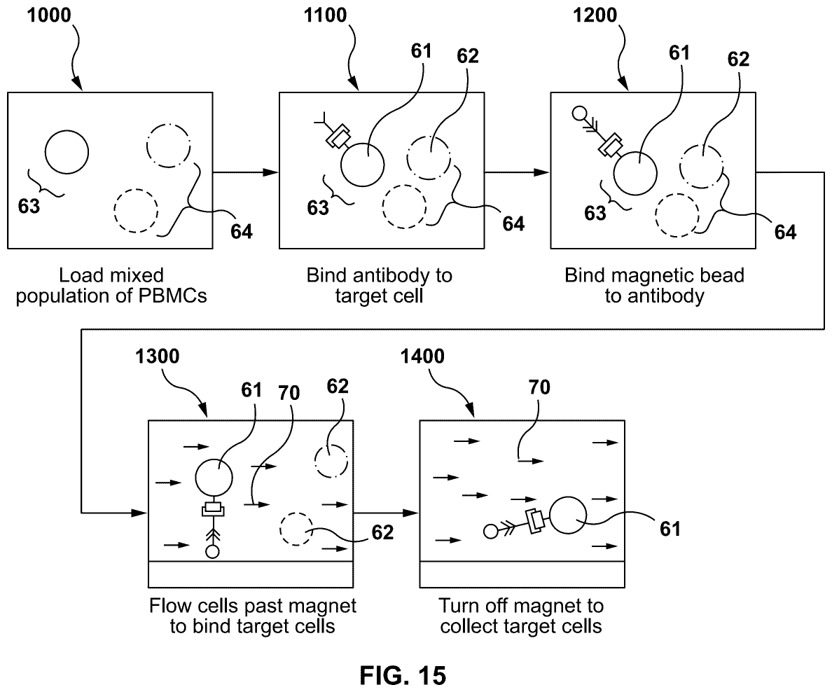

FIG. 15 depicts a method for collecting a biological population from a biological sample using the magnetic separation system 110/210/310/410/510/610/710 as described herein. The method may, for example, include steps such as a loading step 1000, an antibody binding step 1100, a magnetic particle binding step 1200, a magnetic capture step 1300, and/or a collection step 1400. The target biological population for collection from the biological sample may include at least a first subpopulation and a second subpopulation, or a target biological population 61 or non-target biological population 62.

The loading step 1000 may include the loading of the biological sample 60 into the magnetic separation system 110/210/310/410/510/610/710 as previously described herein. In embodiments, the biological sample 60 is a blood cell, white blood cell or platelet. White blood cells (leukocytes) include neutrophils, lymphocytes (T cells inclusive of T helper cells, cytotoxic T cells, T-killer cells, Natural Killer, and B lymphocytes), monocytes, eosinophils, basophils, macrophages, and dendritic cells. In embodiments, the biological sample 60 may include peripheral blood mononuclear cells (PBMC), such as T cells, monocytes, natural killer cells, and/or dendritic cells. The biological sample 60 may include a first subpopulation 63 and a second subpopulation 64, wherein the first subpopulation 63 may include either target cells 61 or non-target cells 62, and the second subpopulation may include the other of the target cells 61 or non-target cells 62.

The antibody binding step 1100 may include the binding of an antibody to a target cell 61. In embodiments, the step 1100 may include the binding of an antibody to a non-target cell 62. In embodiments, the step 1100 may include binding the antibody to cells within the first subpopulation 63 of the biological sample 60. In embodiments, the step 1100 may include binding the antibody to cells within the second subpopulation 64 of the biological sample 60. Binding the antibodies to the cells within the biological sample 60 will enable those antibody-bound cells to bind to the plurality of magnetic particles 40 as described herein.

The magnetic particle binding step 1200 may include binding cells within the biological sample 60 to the plurality of magnetic particles 40. In embodiments, the cells bound to the plurality of magnetic particles 40 are those cells that have first been bound to the antibodies in the antibody binding step 1100. By binding at least a portion of the biological sample 60 to the plurality of magnetic particles 40, the effectiveness of retention of the biological sample 60 to the magnetic field or the magnetic field gradient 52 may be increased once the magnetic field 52 is applied to the fluidic pathway 30 while the biological sample 60 and/or magnetic particles 40 flows through the fluidic pathway 30 along the flow path 70. The separation speed may therefore be increased with more magnetic particles 40 attached to desired cells within the biological sample 60. In embodiments, the magnetic particle binding step 1200 may include binding the first subpopulation 63 of the biological sample 60 to the plurality of magnetic particles 40. The first subpopulation may therefore be subjected to an increased effectiveness of retention to the magnetic field 52 while the first subpopulation 63 flows through the fluidic pathway 30 along the flow path 70, and thus to an increased separation speed between the first subpopulation 63 and the rest of the biological sample 60. In embodiments, the step 1200 may include binding the second subpopulation 64 of the biological sample to the plurality of magnetic particles 40. The second subpopulation 64 may therefore be subjected to an increased effectiveness of retention to the magnetic field 52 while the second subpopulation 64 flows through the fluidic pathway 30 along the flow path 70, and thus to an increased separation speed between the second subpopulation 64 and the rest of the biological sample 60. In embodiments, the magnetic particle binding step 1200 may include binding the target cells 61 of the biological sample 60 to the plurality of magnetic particles 40. In embodiments, the step 1200 may include binding the non-target cells 62 of the biological sample 60 to the plurality of magnetic particles 40.

The magnetic capture step 1300 may include flowing the biological sample 60 through the flow path 70 of fluidic pathway 30. This may further include flowing the biological sample 60 at least partially bound to the plurality of magnetic particles 40 through the flow path 70 of the fluidic pathway 30. In embodiments, the first subpopulation 63 of the biological sample 60 is bound to the plurality of magnetic particles 40. In embodiments, the second subpopulation 64 of the biological sample 60 is bound to the plurality of magnetic particles 40. Flowing the biological sample 60 through the flow path 70 of fluidic pathway 30 may further include flowing the biological sample 60 through or past the entrapment features 34 disposed on or within the fluidic pathway 30. The magnetic capture step 1300 may further include positioning an array of magnets 50 in an ON position such that the fluidic pathway 30 is exposed to a magnetic field or magnetic field gradient 52 generated by the array of magnets 50. Exposing the fluidic pathway 30 to the magnetic field 52 results in exposing the biological population 60 to the magnetic field 52 once the biological population 60 is flowed through the fluidic pathway 30 along the flow path 70.

By flowing the biological population 60 the magnetic field or the magnetic field gradient 52, the portions of the biological population 60 bound to the plurality of magnetic particles 40 may become subjected to a decrease in flow velocity through the fluidic pathway 30, and may further become entrapped within the fluidic pathway 30 via the entrapment features 34 (illustrated in FIG. 14B, for example). The decrease in flow velocity and/or entrapment of the portions of the biological population 60 bound to the magnetic particles 40 results in an increased effectiveness of retention of the portions of the biological population 60 bound to the magnetic particles 40 to the magnetic field 52. In embodiments, the first subpopulation 63 bound to the plurality of magnetic particles 40 is subjected to a decrease in flow velocity, or entrapped to the entrapment features 34 and/or a sidewall 33 of the fluidic pathway as the forces of the magnetic field 52 act upon the magnetic particles 40 and pull the magnetic particles 40, along with the first subpopulation 63 bound to the plurality of magnetic particles 40, into or up against the entrapment features 34 and/or the sidewall 33 of the fluidic pathway 30. While the first subpopulation 63 is decreased in flow velocity or entrapped via the entrapment features 34, the second subpopulation 64, which is not bound to the plurality of magnetic particles 40, may continue to flow through and be removed from the fluidic pathway 30 along the flow path 70. In embodiments, the second subpopulation 64 bound to the plurality of magnetic particles 40 is subjected to a decrease in flow velocity, or entrapped to the entrapment features 34 and/or a sidewall 33 of the fluidic pathway as the forces of the magnetic field 52 act upon the magnetic particles 40 and pull the magnetic particles 40, along with the second subpopulation 64 bound to the plurality of magnetic particles 40, into or up against the entrapment features 34 and/or the sidewall 33 of the fluidic pathway 30. While the second subpopulation 64 is decreased in flow velocity or entrapped via the entrapment features 34, the first subpopulation 63, which is not bound to the plurality of magnetic particles 40, may continue to flow through and be removed from the fluidic pathway 30 along the flow path 70.

In embodiments, the biological population 60 comprises a target biological population 61 and a non-target biological population 62. It should be understood that the plurality of magnetic particles 40 may be configured to bind with either of the target biological population 61 and/or the non-target biological population 62 as previously described herein. In embodiments, the first subpopulation 63 comprises the target biological population 61, and the second subpopulation 64 comprises the non-target biological population 62. In embodiments, the first subpopulation 63 comprises the non-target biological population 62, and the second subpopulation 64 comprises the target biological population 61.

The collection step 1400 may include positioning the array of magnets 50 in an OFF position such that the fluidic pathway 30, and/or the biological population 60 entrapped within the fluidic pathway 30 is not exposed to the magnetic field or the magnetic field gradient 52. In embodiments, the array of magnets 50 is swiveled away from, or translated away from, the fluidic pathway 30, such that the distance between the array of magnets 50 and the biological population 60/magnetic particles 40 is increased, as previously described across the various magnetic separation systems 110/210/310/410/510/610/710 presented herein. Once the biological population 60 is no longer exposed to the magnetic field 52, the portion of the biological population 60 bound to the plurality of magnetic particles 40 may be removed from or flowed out of the fluidic pathway 30 for collection. In embodiments, the collection step 1400 may include increasing the flow rate of the biological population 60 through the fluidic pathway 30. In embodiments, the first subpopulation 63 bound to the plurality of magnetic particles 40 may be removed or flowed out of the fluidic pathway 30 for collection. In embodiments, the second subpopulation 64 bound to the plurality of magnetic particles 40 may be removed or flowed out of the fluidic pathway 30 for collection.

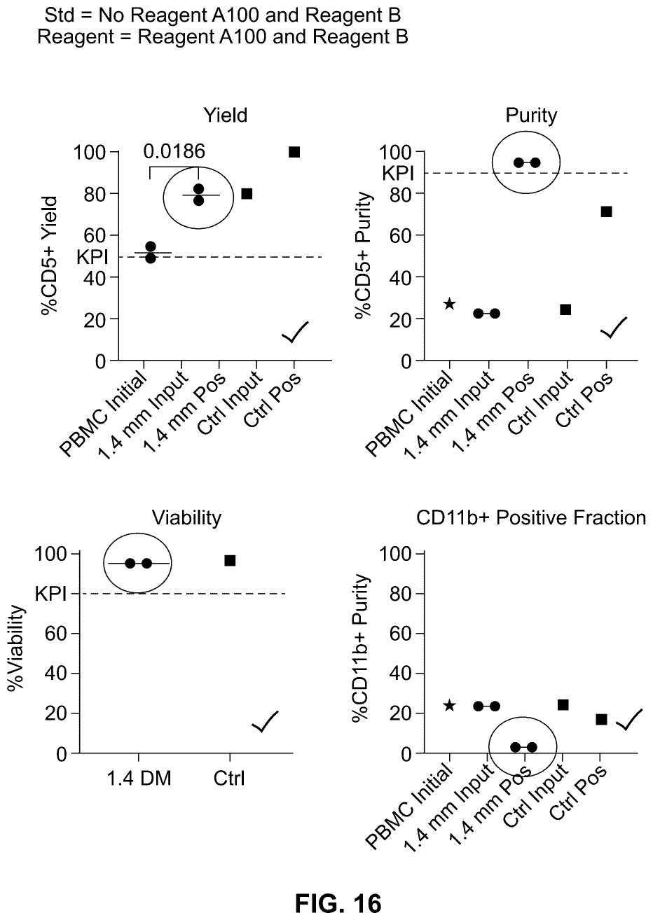

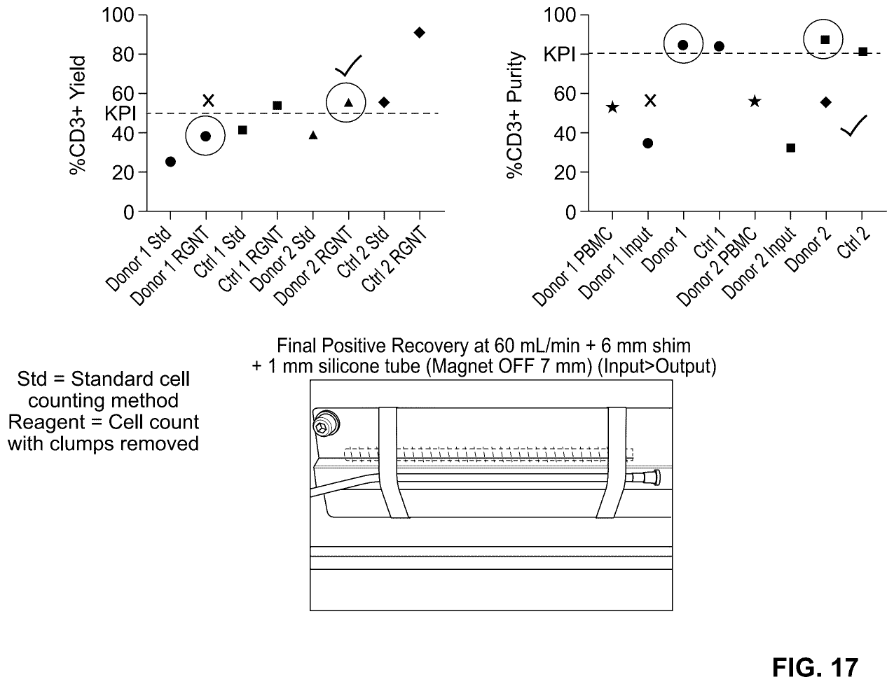

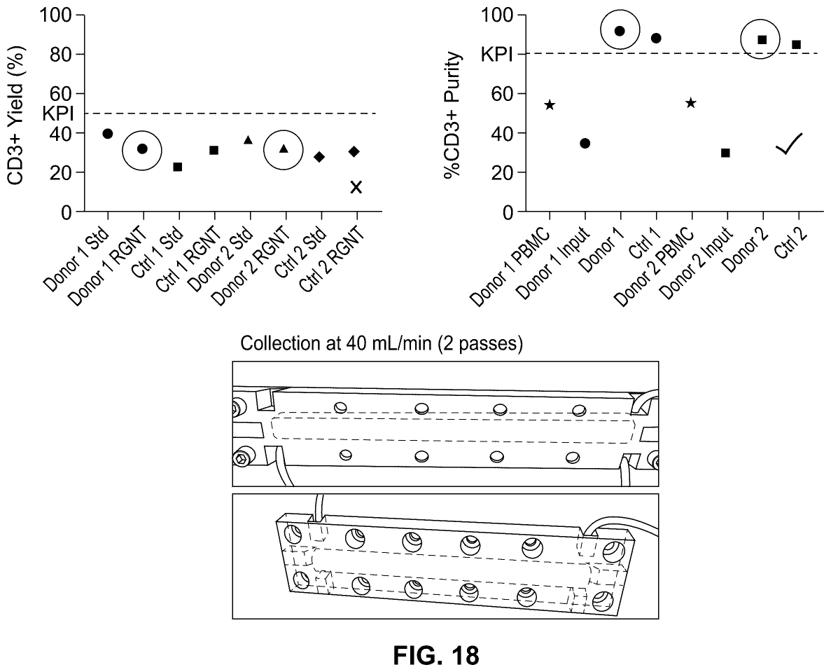

FIGS. 16-18 provide test results demonstrating the improvements of the magnetic separation system 110/210/310/410/510/610/710 in variables such as cell yield, purity, and viability, among other parameters. In particular, FIG. 16 illustrates the improved yield, purity, and viability results of nanobead magnetic particle performance with the flow-through tubing or fluidic pathway 30 aspects of the invention, demonstrating the high-qualitative and/or high-quantitative separation of target cells from a biological population. FIG. 17 illustrates the improved yield and purity results of micron-sized beads magnetic particle separation performance when the array of magnets of the present invention are retrofitted onto prior magnetic separation systems, demonstrating the high-qualitative and/or high-quantitative separation of target cells from a biological population. FIG. 18 illustrates the improved yield and purity results of micron-sized beads magnetic particle separation performance when the fluidic pathway 30 and/or array of magnets of the present invention are applied, demonstrating the high-qualitative and/or high-quantitative separation of target cells from a biological population.

### EXAMPLES

Embodiment 1. A system for magnetic separation and collection of a target biological population from a biological sample, comprising: a cell engineering cassette; a fluidic pathway disposed within the cell engineering cassette, the fluidic pathway having entrapment features disposed along a flow path of the fluidic pathway; and an array of magnets disposed adjacent to the fluidic pathway and to the cell engineering cassette such that the array of magnets can be translatable toward and away from the fluidic pathway.

Embodiment 2. The system of embodiment 1, wherein the array of magnets is configured as a Halbach array.

Embodiment 3. The system of embodiment 1 or embodiment 2, wherein the fluidic pathway is configured such that a height of the fluidic pathway is greater than a width of the fluidic pathway, to allow for a larger capture volume therein.

Embodiment 4. The system of embodiments 1-3, wherein the entrapment features are square, triangular, semicircular or semioval protrusions into the flow path of the fluidic pathway.

Embodiment 5. The system of embodiments 1-4, wherein the entrapment features are square, rectangular, triangular, semicircular, or semioval formations formed in a sidewall of the fluidic pathway along the flow path.

Embodiment 6. The system of embodiments 1-5, wherein the array of magnets is disposed adjacent a satellite bag, a proliferation chamber, a crossflow reservoir, an input module of the cell engineering cassette, or a warm zone, and/or a cold zone of the system for magnetic separation.

Embodiment 7. The system of embodiments 1-6, wherein the entrapment features are formed substantially perpendicular to the flow path of the fluidic pathway.

Embodiment 8. The system of embodiments 1-7, wherein the entrapment features are formed at an angle substantially between 0°-90° to the flow path of the fluidic pathway.

Embodiment 9. The system of embodiments 1-8, wherein the entrapment features are formed at an angle substantially 45° to the flow path of the fluidic pathway.

Embodiment 10. The system of embodiments 1-9, wherein the fluidic pathway is configured for multi-directional flow of the flow path.

Embodiment 11. The system of embodiments 1-10, wherein the fluidic pathway is configured for one or more passes of the biological sample through the fluidic pathway along the flow path.

Embodiment 12. The system of embodiments 1-11, wherein the entrapment features of the fluidic pathway are configured to decrease a flow velocity of a plurality of magnetic particles moving through the flow path.

Embodiment 13. The system of embodiment 12, wherein the decreased flow velocity allows for a decreased shear stress and fluid drag on the plurality of magnetic particles.

Embodiment 14. The system of embodiments 1-13, wherein the entrapment features of the fluidic pathway generate a shear stress between 0 to 1 Pascals along the flow path.

Embodiment 15. The system of embodiments 1-14, wherein each individual entrapment feature of the entrapment features of the fluidic pathway has a width of approximately 0.05-1 mm.

Embodiment 16. The system of embodiments 1-15, wherein the entrapment features are configured to encumber or retain a plurality of magnetic particles as they move along the flow path of the fluidic pathway.

Embodiment 17. The system of embodiment 16, wherein each of the plurality of magnetic particles have a diameter between 50 nm to 5 μm.

Embodiment 18. A method for collecting a biological population from a biological sample having at least a first subpopulation and a second subpopulation, comprising: binding the first subpopulation to a plurality of magnetic particles; flowing the biological sample through a flow path of a fluidic pathway having entrapment features disposed therein; positioning an array of magnets such that the fluidic pathway is exposed to a magnetic field generated by the array of magnets; exposing the biological population to the magnetic field; entrapping the first subpopulation bound to the plurality of magnetic particles to the entrapment features and/or a sidewall of the fluidic pathway; removing and collecting the second subpopulation from the fluidic pathway; positioning the array of magnets such that the fluidic pathway is not exposed to the magnetic field; removing the first subpopulation bound to the plurality of magnetic particles from the fluidic pathway; and collecting the first subpopulation bound to the plurality of magnetic particles.

Embodiment 19. The method of embodiment 18, further including decreasing a flow velocity of the first subpopulation through the flow path of the fluidic pathway via the entrapment features.

Embodiment 20. The method of embodiment 18 or embodiment 19, further decreasing a shear stress and fluid drag on the first subpopulation by decreasing the flow velocity of the first subpopulation through the flow path via the entrapment features.

Embodiment 21. The method of embodiments 18-20, further including arranging the array of magnets in a Halbach array.

Embodiment 22. The method of embodiments 18-21, further including flowing the biological sample through the fluidic pathway in a multi-directional flow.

Embodiment 23. The method of embodiments 18-22, further including flowing the biological sample through the fluidic pathway in a single directional flow.

Embodiment 24. The method of embodiments 18-23, wherein the first subpopulation comprises a target biological population within the biological sample, and the second subpopulation comprises a non-target biological population within the biological sample.

Embodiment 25. The method of embodiments 18-24, wherein the first subpopulation comprises a non-target biological population within the biological sample, and the second subpopulation comprises a target biological population within the biological sample.

Embodiment 26. The method of embodiments 18-25, wherein removing the first subpopulation bound to the plurality of magnetic particles from the fluidic pathway further includes at least one of the following steps: (a) increasing distance between the array of magnets and the plurality of magnetic particles; (b) pivoting the array of magnets away from the plurality of magnetic particles to divert a direction of the magnetic field; (c) increasing a flow rate of the biological sample through the flow path; (d) placing a shield or barrier between the array of magnets and the plurality of magnetic particles; and (e) using a combined arrangement of the array of magnets such that the array of magnets include a Halbach array and an alternating array, wherein the Halbach array is inline with the flow path.

Embodiment 27. The method of embodiments 18-26, further including the step of adding antibodies to the biological population prior to the step of binding the first subpopulation to a plurality of magnetic particles.

Embodiment 28. A fluidic pathway for flowing a biological sample and magnetic particles along a flow path therein, comprising: a fluidic pathway having a height of approximately 5-44 mm, a width of approximately 0.5-6 mm, and a length of approximately 50-520 mm; and entrapment features disposed along the length of the fluidic pathway, each of the entrapment features have a height and/or a width of about 0.05-1 mm; wherein the entrapment features are configured to decrease a flow velocity of a first subpopulation of the biological sample, wherein the first subpopulation is bound to a plurality of magnetic particles.

Embodiment 29. The fluidic pathway of embodiment 28, wherein the entrapment features are square, triangular, semicircular or semioval protrusions into the flow path of the fluidic pathway.

Embodiment 30. The fluidic pathway of embodiment 28 or embodiment 29, wherein the entrapment features are formed as square, rectangular, triangular, semicircular, or semioval formations formed in a sidewall of the fluidic pathway along the flow path.

Embodiment 31. The fluidic pathway of embodiments 28-30, wherein the entrapment features are formed substantially perpendicular to the flow path of the fluidic pathway.

Embodiment 32. The fluidic pathway of embodiments 28-31, wherein the entrapment features are formed at an angle substantially between 0°-90° to the flow path of the fluidic pathway.

Embodiment 33. The fluidic pathway of embodiments 28-32, wherein the entrapment features are formed at an angle of approximately 45° to the flow path of the fluidic pathway.

Embodiment 34. The fluidic pathway of embodiments 28-33, wherein the fluidic pathway is configured for multi-directional flow of the flow path.

Embodiment 35. The fluidic pathway of embodiments 28-34, wherein the entrapment features of the fluidic pathway generate a shear stress between 0 to 1 Pascals along the flow path.

Embodiment 36. The fluidic pathway of embodiments 28-35, wherein each of the entrapment features are spaced apart approximately 1 mm to 5 mm from an adjacent entrapment feature.

Embodiment 37. The fluidic pathway of embodiments 28-36, wherein approximately 20-200 entrapment features are disposed along the length of the fluidic pathway.

Embodiment 38. The fluidic pathway of embodiments 28-37, wherein the height of the fluidic pathway is approximately 16 mm.

Embodiment 39. The fluidic pathway of embodiments 28-38, wherein the width of the fluidic pathway is approximately 2 mm.

Embodiment 40. The fluidic pathway of embodiments 28-39, wherein the length of the fluidic pathway is approximately 180 mm.

Embodiment 41. The fluidic pathway of embodiments 28-40, wherein the height and/or width of each of the entrapment features is approximately 0.3 mm.

Embodiment 42. A system for magnetic separation and collection of a target biological population from a biological sample, comprising: a cell engineering cassette; a fluidic pathway disposed within the cell engineering cassette; and an array of magnets disposed adjacent to the fluidic pathway and to the cell engineering cassette, the array of magnets configured for engaging the fluidic pathway with a magnetic field when in an ON position, and configured for disengaging, disrupting, or blocking the magnetic field from the fluidic pathway when in an OFF position.

Embodiment 43. The system of embodiment 42, wherein the array of magnets is disposed adjacent a satellite bag, a proliferation chamber, a crossflow reservoir, an input module of the cell engineering cassette, or a warm zone, and/or a cold zone of the system for magnetic separation.

1. A system for magnetic separation and collection of a target biological population from a biological sample, comprising:
a cell engineering cassette;
a fluidic pathway disposed within the cell engineering cassette, the fluidic pathway having entrapment features disposed along a flow path of the fluidic pathway; and
an array of magnets disposed adjacent to the fluidic pathway and to the cell engineering cassette such that the array of magnets can be translatable toward and away from the fluidic pathway.

2. The system of claim 1, wherein the array of magnets is configured as a Halbach array.

3. The system of claim 1, wherein the fluidic pathway is configured such that a height of the fluidic pathway is greater than a width of the fluidic pathway, to allow for a larger capture volume therein.

4. The system of claim 1, wherein the entrapment features are square, triangular, semicircular or semioval protrusions into the flow path of the fluidic pathway.

5. The system of claim 1, wherein the entrapment features are square, rectangular, triangular, semicircular, or semioval formations formed in a sidewall of the fluidic pathway along the flow path.

6. The system of claim 1, wherein the array of magnets is disposed adjacent a satellite bag, a proliferation chamber, a crossflow reservoir, an input module of the cell engineering cassette, or a warm zone, and/or a cold zone of the system for magnetic separation.

7. The system of claim 1, wherein the entrapment features are formed substantially perpendicular to the flow path of the fluidic pathway.

8. The system of claim 1, wherein the fluidic pathway is configured for multi-directional flow of the flow path.

9. The system of claim 1, wherein the fluidic pathway is configured for one or more passes of the biological sample through the fluidic pathway along the flow path.

10. The system of claim 1, wherein the entrapment features of the fluidic pathway are configured to decrease a flow velocity of a plurality of magnetic particles moving through the flow path.

11. The system of claim 1, wherein the entrapment features are configured to encumber or retain a plurality of magnetic particles as they move along the flow path of the fluidic pathway.

12. A method for collecting a biological population from a biological sample having at least a first subpopulation and a second subpopulation, comprising:
binding the first subpopulation to a plurality of magnetic particles;
flowing the biological sample through a flow path of a fluidic pathway having entrapment features disposed therein;
positioning an array of magnets such that the fluidic pathway is exposed to a magnetic field generated by the array of magnets;
exposing the biological population to the magnetic field;
entrapping the first subpopulation bound to the plurality of magnetic particles to the entrapment features and/or a sidewall of the fluidic pathway;
removing and collecting the second subpopulation from the fluidic pathway;
positioning the array of magnets such that the fluidic pathway is not exposed to the magnetic field;
removing the first subpopulation bound to the plurality of magnetic particles from the fluidic pathway; and
collecting the first subpopulation bound to the plurality of magnetic particles.

13. The method of claim 12, wherein removing the first subpopulation bound to the plurality of magnetic particles from the fluidic pathway further includes at least one of the following steps:
(a) increasing distance between the array of magnets and the plurality of magnetic particles;
(b) pivoting the array of magnets away from the plurality of magnetic particles to divert a direction of the magnetic field;
(c) increasing a flow rate of the biological sample through the flow path;
(d) placing a shield or barrier between the array of magnets and the plurality of magnetic particles; and
(e) using a combined arrangement of the array of magnets such that the array of magnets include a Halbach array and an alternating array, wherein the Halbach array is inline with the flow path.

14. A fluidic pathway for flowing a biological sample and magnetic particles along a flow path therein, comprising:
a fluidic pathway having a height of approximately 5-44 mm, a width of approximately 0.5-6 mm, and a length of approximately 50-520 mm; and
entrapment features disposed along the length of the fluidic pathway, each of the entrapment features have a height and/or a width of about 0.05-1 mm;
wherein the entrapment features are configured to decrease a flow velocity of a first subpopulation of the biological sample, wherein the first subpopulation is bound to a plurality of magnetic particles.

15. The fluidic pathway of claim 14, wherein the entrapment features are square, triangular, semicircular or semioval protrusions into the flow path of the fluidic pathway.

16. The fluidic pathway of claim 14, wherein the entrapment features are formed as square, rectangular, triangular, semicircular, or semioval formations formed in a sidewall of the fluidic pathway along the flow path.

17. The fluidic pathway of claim 14, wherein the entrapment features are formed substantially perpendicular to the flow path of the fluidic pathway.

18. The fluidic pathway of claim 14, wherein the fluidic pathway is configured for multi-directional flow of the flow path.

19. A system for magnetic separation and collection of a target biological population from a biological sample, comprising:
a cell engineering cassette;
a fluidic pathway disposed within the cell engineering cassette; and
an array of magnets disposed adjacent to the fluidic pathway and to the cell engineering cassette, the array of magnets configured for engaging the fluidic pathway with a magnetic field when in an ON position, and configured for disengaging, disrupting, or blocking the magnetic field from the fluidic pathway when in an OFF position.

20. The system of claim 19, wherein the array of magnets is disposed adjacent a satellite bag, a proliferation chamber, a crossflow reservoir, an input module of the cell engineering cassette, or a warm zone, and/or a cold zone of the system for magnetic separation.
